(19) United States
(12) Patent Application Publication
(10) Pub. No.: US 2007/0134662 A1
(43) Pub. Date: Jun. 14, 2007

(54) Structural interaction fingerprint

(76) Inventors: Juswinder Singh, Ashland, MA (US); Claudio Chuaqui, Arlington, MA (US); Zhan Deng, Winchester, MA (US)

Correspondence Address: STEPTOE & JOHNSON LLP, 1330 CONNECTICUT AVE., NW, WASHINGTON, DC, 20036, US

(21) Appl. No.: 10/562,974

(22) PCT Filed: Jul. 1, 2004
(86) PCT No.: PCT/US04/20992

§ 371 Date: Jul. 31, 2006

Related U.S. Application Data
(60) Provisional application No. 60/484,308, filed on Jul. 3, 2003.
(60) Provisional application No. 60/524,083, filed on Nov. 24, 2003.

(51) Int. Cl. C12Q 1/68; G01N 33/53; G06F 19/00

(52) U.S. Cl. 435/6; 435/7.1; 702/20

(57) ABSTRACT

Disclosed is a method for representing and analyzing 3D target molecule-ligand intermolecular interactions. The method generates structural interaction fingerprints (SIFts) that convert three-dimensional structural interaction information into linear information strings that contains a plurality of information blocks; each of which in turn containing a plurality of information units. By assigning to each information unit a calculated value to represent the characteristic of a set of intermolecular interactions occurring at each selected position (i.e., a position on the target molecule at which intermolecular interaction occurs), a SIFt of the target molecule-ligand complex is constructed.

### PRIORITY CLAIM

This application claims priority under 35 U.S.C. § 119(e) to U.S. patent application Ser. No. 60/524,083, filed Nov. 24, 2003, and U.S. patent application Ser. No. 60/484,308, filed Jul. 3, 2003, each of which is incorporated by reference in its entirety.

### BACKGROUND

Representing and understanding the three-dimensional structural information of biological molecules is becoming a critical step in the rational drug discovery process. With the advent of massive virtual chemical library screening, as well as the recent advancements in X-ray crystallography, NMR and homology modeling techniques, the amount of structural information increases at an explosive speed. The traditional analysis methods are inadequate and inefficient in dealing with such massive structural information.

The past decade has seen an explosion of the three-dimensional structural information of biologically important molecules, thanks to the recent developments of X-ray crystallography, NMR and molecular modeling techniques. There are currently about 20,000 holdings deposited in the Protein Data Bank, and a significant portion of these structures contain ligands bound to macromolecules. In addition, combinatorial chemistry and virtual library screening are becoming routine procedures in the drug discovery process. This process generates thousands to millions of virtual protein-ligand complex structures, making detailed examination of these structures a daunting task. Representing the three-dimensional structural information of macromolecules has always been a challenge due to the complexity of identifying residues and atomic interactions. Representing the covalent or non-covalent interactions between molecules poses even more difficult challenges, because not only is the geometric location of each interaction needed, but also the direction, type, and magnitude of the interaction are also important and need to be captured. Understanding the intermolecular interactions between proteins and their ligands is of great importance as it provides insights into the functional mechanism of the proteins. It is important for structure-based drug design to understand the key forces between small molecules (SMs) and proteins and to be able to compare different orientations or different small molecules binding to the same receptor site, or different binding sites.

Traditionally, understanding and comparing the interactions between proteins and ligands is achieved by visually inspecting individual structure with structure-rendering software on a graphic terminal, sometimes facilitated by other software tools that generate 2-D or 3-D schematic representations of the interactions (e.g., LIGPLOT®). Such time-consuming processes require human intervention and it becomes more and more tedious as the number of complex structures increases. It is important for successful drug discovery to have a tool that allows this massive amount of structural information to be organized and analyzed.

More recently, structure-based virtual chemical library screening has become a common procedure in the drug discovery process. Virtual library screening typically generates hundreds of thousands of virtual protein-ligand complex structures. Effectively mining this massive structural library becomes a tremendous task, as it is impossible to analyze the structures individually. Traditionally, different types of empirical docking scores and some pharmacophoric filters are used to sift the docking results for tight binders with desired binding interactions. However, these methods have limitations. Correlation between good docking scores and high activity is not always satisfactory. The docking scores are an overall summation of interaction and do not discern differences in binding modes. Therefore, a method that allows accurate representation of the interaction and fast analysis of a large number of structures is in great demand.

### SUMMARY

In one aspect, a method is provided for generating a structural interaction fingerprint (SIFt). The SIFt is in the form of an information string which includes a plurality of information blocks, and each information block includes a plurality of information units. The method includes the steps of selecting a plurality of positions (selected positions) on a target molecule where each selected position corresponds to an information block in the information string; selecting a plurality of interaction types and calculating a value that is indicative of the characteristic of each interaction type at each selected position of the target molecule; assigning the value to the corresponding information unit thereby indicating the characteristic of that particular interaction type at the corresponding selected position; and joining the information units of each selected position together to form the corresponding information blocks, which joins together to generate a SIFt.

The target molecule can be a protein or a fragment thereof, such as a peptide (e.g., polypeptide or oligopeptide). Alternatively, a target molecule can be a nucleic acid. In certain circumstances, the ligand can be a peptide, a nucleic acid, or even a small molecule (e.g., an organic molecule (e.g., molecular weight equal to or less than 1,500 dalton) that is neither a peptide or a nucleic acid).

Note that the target molecule is forming a complex with a ligand (i.e., the binary complex), and the selected positions are the positions on the target molecule that participate in intermolecular interaction with the ligand. These positions can be obtained from a three-dimensional structure of a binary complex formed between the target molecule and the ligand. The three-dimensional structure can be derived from an experimental method or a prediction method such as, for example, an in silico prediction method. In one embodiment, a set of selected positions can be obtained from comparing the common positions (e.g., residues or bases) of the target molecule that participate in intermolecular interactions among a set of target molecule-ligand structures. The target molecule can be the same or different in the set of target molecule-ligand structures.

For a protein or peptide target molecule, each selected position can include one or more secondary structure elements (e.g., an α-helix or a β-strand), amino acid residues (e.g., a lysine residue), main chain atom groups (the α-carbon of a particular amino acid residue), side chain atom groups (e.g., the butylamine group of a Lys), or individual atoms of the target molecule. As to a nucleic acid target molecule, each selected position can include one or more bases, functional groups, or individual atoms of the target molecule.

The value that is assigned to a particular information unit can be a binary value or a numeric value selected from a scale or range of numbers. The binary value indicates whether a particular interaction type is present (1) or absent (0) at the corresponding selected position of the target molecule, whereas the numeric value indicates the magnitude of a particular interaction type at the corresponding selected position of the target molecule (e.g., a value of “3” in a scale that ranges from “0” to “5”).

As mentioned above, the value indicates the characteristic of a particular interaction type at that selected position. Note that the interaction types represent different types of intermolecular interactions between the target molecule and the ligand. For example, the interaction type can be classified as contact interaction. One can detect the presence of contact interaction between a target molecule and a ligand at a selected position (e.g., a protein residue) according to a number of methods. In one embodiment, the target molecule-ligand pair is considered to have established contact interaction at a selected position if the interaction involves a change or reduction in the accessible surface area at that position of the target molecule upon forming a complex with the ligand. Alternatively, one can measure the intermolecular distance between a target molecule and a ligand at a selected position to determine whether contact interaction occurs at that position (i.e., whether the intermolecular distance is within the predetermined distance cutoff limit). In one embodiment, the target molecule-ligand pair is considered to be interacting if the interatomic contact distance between the target molecule and the ligand is equal to or less than 10 Å (e.g., equal to or less than 6 Å, or even 4 Å). The interaction type can be further classified as polar interaction, non-polar interaction, and/or hydrogen bonding interaction, depending on the nature of the interactions. In one embodiment, the hydrogen bonding interaction can involve a hydrogen bond donor in the target molecule and a hydrogen bond acceptor in the ligand at the selected position. In one embodiment, the hydrogen bonding interaction can involve a hydrogen bond acceptor in the target molecule and a hydrogen bond donor in the ligand at the selected position. Note that intermolecular interactions can be characterized by interaction energy-based approach. The interaction type can be characterized by the contribution of the selected position to the interaction energy between a target molecule and a ligand where the total interaction energy between the target and the ligand is a summed over all positions. The interaction energy may be computed by a variety of scoring functions or intermolecular force-fields such as common ligand-receptor docking scoring functions (e.g., Dock, Gold, ChemScore, FlexX score, PMF, Screencore, Drugscore, etc.) or intermolecular potential energy functions or force-fields (e.g., CHARMM, Amber, OPLS, etc.). The interaction energy calculated for each information unit (which corresponds to a selected position) may take the form of a real number (i.e., −43.2 kcal/mol), integer (i.e., −43 kcal/mol), or an integer representing a binned form of the interaction energy. In the latter case, the energy range of the function is divided into bins (e.g., [−70 to −50 kcal/mol], [−50 to −20 kcal/mol], [−20 to 0 kcal/mol], or [0-10 kcal/mol]) where the interaction energy is represented as an integer identifying the bin (in this case for example 1, 2, 3, or 4).

In one aspect, a method of predicting the interaction pattern between a target molecule and a test ligand is provided. A test ligand is a ligand whose affinity to the target molecule is under examination. The prediction method involves identifying a plurality of selected positions between the target molecule and a first ligand, wherein the first ligand is known to bind to the target molecule (i.e., the affinity between the first ligand and the target molecule is known). As described above, selected positions are positions on the target molecule that participate in intermolecular interactions with the ligand (here, the first ligand). Based on the selected positions, the method then involves generating a first structural interaction fingerprint (SIFt) as described above (i.e., formation of an information string that includes a plurality of information blocks, where each information block includes a plurality of information units, and where each information unit is assigned a calculated value indicative of the presence/absence or the magnitude of a particular interaction type at the selected position of the target molecule to which the information unit/block corresponds). Using the same selected positions, the method then involves the generation of a second SIFt between the same target molecule and a second ligand (i.e., a test ligand) employing the same steps as described above. Finally, the method involves comparing the first SIFt with the second SIFt to determine the level of overlapping between the first and second SIFts. A pattern of substantial overlapping between the two SIFts predicts that the second ligand interacts with the target molecule in a similar pattern as the first ligand. In one embodiment, the first ligand is the natural ligand of the target molecule. In one embodiment, the first ligand is a ligand of known affinity to the target molecule.

In one aspect, a method of generating a structural interaction fingerprint (SIFt) database is provided. The method involves (1) identifying a plurality of selected positions on a target molecule (which forms a complex with a first ligand) and (2) generating a first SIFt of the database as described above (i.e., formation of an information string that includes a plurality of information blocks where each information block includes a plurality of information units, and where each information unit is assigned a calculated value indicative of the presence/absence or the magnitude of a particular interaction type at the selected position of the target molecule to which the information unit/block corresponds). The method then requires that steps (1) and (2) be repeated using the same target molecule but a different ligand such that another SIFt can be generated and added to the databases. The method then repeats steps (1) and (2) with different ligands and generates more SIFts until the database contains a desired number of SIFts. In one embodiment, the method further involves analyzing the SIFts of the database to generate one or more interaction patterns between the target molecule and the ligands. Typically, ligands that belong to a particular interaction pattern indicate that they bind to the target molecule in a similar manner. In one embodiment, the method further involves comparing one (or more) interaction pattern of the database with a SIFt generated by using the same target molecule and a test ligand. A test ligand is a ligand that was not employed in generating the database. From the degree of similarity between the SIFt generated using the test ligand and the interaction pattern, one can predict whether or not the test ligand binds to the target molecule in a similar manner. One can even predict whether or not the test ligand belongs to the same family of ligands used to generate the database. In one embodiment, the method further includes the step of storing the database in a computer readable medium.

In one aspect, a method of analyzing the interaction pattern of two or more related target molecules is provided. The method includes conducting sequence and structural alignments among each of the related target molecules resulting to derive a uniform residue or base numbering system. The method then involves identifying a plurality of selected positions on the target molecule of each target molecule-ligand complex using the uniform residue or base numbering system. This is followed by generating a SIFt for each target molecule-ligand complex as described above and comparing different SIFt patterns. The interactions can be conserved or unconserved.

The method can include compiling the SIFts to identify selected interactions that are conserved among the complexes. The method can include calculating a score for each interaction among the target molecule-ligand complexes. The score can include a conservation score. The method can include compiling the SIFts to form an interaction profile from the calculated conservation score, or comparing a SIFt generated from a test ligand with an interaction profile generated from a group of target molecule-ligand complexes, thereby predicting whether the test ligand interacts with the target molecule in a similar pattern with the group. The method can include comparing two interaction profiles, thereby predicting whether two groups of structures share conserved binding interactions, and/or have similar binding pattern.

As used herein, the target molecules are related if they exhibit at least 20% sequence similarity or a structural similarity with a root-mean squared deviation over the aligned positions no greater than 4 Å (e.g., 6 Å). In yet another embodiment, the target molecules are related if they exhibit at least 20% protein sequence similarity with a root-mean squared deviation over the aligned positions no greater than 6 Å. For protein target molecules, sequence and structural alignments are commonly applied within the structural biology field. There are databases including the PFAM database that includes protein sequence alignments (http://www.sanger.ac.uk/software/Pfam/index.shtml) and the SCOP database (http://scop.mrc-lmb.cam.ac.uk/scop/) that contains protein structural alignments.

In some embodiments, at least one interaction type includes a chemical or physical property of a part of ligand interacting with each selected position. In other embodiments, each interaction type includes a chemical and physical property of a part of ligand interacting with each selected position. The interaction types can include information bits about the chemical composition of a ligand (e.g., various R groups in a combinatorial library), or an experimentally determined or computed property of the part of the ligand interacting with the selected position. For example, interaction types can include information bits representing varying groups of a combinatorial library. Properties and descriptors of a molecule or part of a molecule can include fragment constant descriptors (e.g., hydrophobic, hydrogen bond acceptor, hydrogen bond donor, hydrophobic aliphatic, hydrophobic aromatic, negative charge, negative ionizible, positive charge, positive ionizible, or aromatic ring), electronic descriptors (e.g., charge, partial positive surface area, partial negative surface area, dipole moment, atomic polarizability, polar surface area), topological descriptors (e.g., Wiener index, Zagreb index, Hosoya index), molecular flexibility index, spatial descriptors (e.g., shadow indices, molecular surface area, density, principal moment of inertia, molecular volume), structural descriptors (e.g., number of chiral centers, molecular weight, number of rotatable bonds), or thermodynamic descriptors (e.g., partition coefficient, desolvation free energies for water and octanol, pKa). The interaction type can also include a chemical fingerprint for a part of the ligand interacting with the selected position of the target molecule. A chemical fingerprint is a string of values (usually an array of binary bits) that contains the unique information about the chemical makeup (e.g., atoms, substructures, chirality) of the molecule. In some embodiments, the interaction types can also include information about the selected position in the target molecule, such as variables measuring the sequence conservation, structural conservation and flexibility of the selected position of the target molecule.

In a further aspect, a computer-readable data storage medium is provided. The medium includes a data storage material encoded with a computer-readable database. The database includes a plurality of SIFts generated from a target molecule and a plurality of ligands. Each SIFt is in the form of an information string that includes a plurality of information blocks, and each information block includes a plurality of information units. The target molecule interacts with each ligand at a plurality of selected positions on the target molecule via a number of interaction types. As described above, selected positions are positions on the target molecule that participate in intermolecular interaction with the ligand. The magnitude of each interaction type at each selected position is calculated and represented by a value, which is assigned to a corresponding information unit. The target molecule a be a protein, a peptide, or a nucleic acid, and the ligand can be a small molecule, a peptide, a protein or a nucleic acid. In one embodiment, the value that is assigned to an information unit is a binary value, which indicates the presence or absence of a particular interaction type at the corresponding selected position. In one embodiment, the value that is assigned to an information unit is selected from a range of scaled numeric values, which indicates the magnitude of a particular interaction type at the corresponding selected position. For a protein/peptide target molecule, each selected position can include one or more amino acid residues, main chain atom groups, side chain atom groups, or individual atoms of the target molecule. For a nucleic acid target molecule, each selected position can include one or more bases, functional groups, or individual atoms of the target molecule. In one embodiment, the interaction type can be a contact interaction. For example, the interatomic contact distance between the target molecule and the ligand can be equal or less than 10 Å (e.g., equal or less than 6 Å, or even 4 Å) for the target molecule-ligand pair to be considered as having contact interaction. As another example, the contact interaction can include a change in the accessible surface area of the target molecule upon forming a complex with the ligand. In one embodiment, the interaction type can be a polar interaction, non-polar interaction, and hydrogen bond interaction. In one embodiment, the hydrogen bond interaction can include a hydrogen bond donor in the target molecule and a hydrogen bond acceptor in the ligand at the corresponding selected position. In one embodiment, the hydrogen bond interaction can include a hydrogen bond acceptor in the target molecule and a hydrogen bond donor in the ligand at the corresponding selected position.

In yet a further aspect, a computer program for generating a SIFt that is in the form of an information string comprising a plurality of information blocks, where each information block includes a plurality of information units is provided. The computer program contains instructions for causing a computer system to select a plurality of positions (selected positions) on a target molecule (which is forming a complex with a ligand). The selected positions are positions on the target molecule that participate in intermolecular interaction with the ligand. Each selected position corresponds to an information block in the information string. The computer program can perform one or more of the following steps: select a plurality of interaction types that exist between the target molecule and the ligand; calculate a value that is indicative of the characteristic of each interaction type at each selected position of the target molecule; assign the value to the corresponding information unit so as to indicate the characteristic of that particular interaction type at the corresponding selected position; join the information units of each selected position together to form the corresponding information blocks; and join the information blocks to generate a SIFt. The target molecule can be a protein, a peptide, or a nucleic acid, and the ligand can be a small molecule, a peptide, or a nucleic acid. In one embodiment, the value that is assigned to an information unit is a binary value, which indicates the presence or absence of a particular interaction type at the corresponding selected position. In one embodiment, the value that is assigned to an information unit is selected from a range of scaled numeric values, which indicates the magnitude of a particular interaction type at the corresponding selected position. In one embodiment, the selected positions are obtained from a three-dimensional structure of a binary complex formed between the target molecule and the ligand. Such a three-dimensional structure may be derived from an experimental method or a prediction method such as, for example, an in silico prediction method. For a protein/peptide target molecule, each selected position can include one or more amino acid residues, main chain atom groups, side chain atom groups, or individual atoms of the target molecule. For a nucleic acid target molecule, each selected position can include one or more bases, functional groups, or individual atoms of the target molecule. The interaction types represent different types of intermolecular interactions between the target molecule and the ligand and can be characterized by binding energy-based approach. In one embodiment, the interaction type can be a contact interaction. For example, the interatomic contact distance between the target molecule and the ligand can be equal or less than 10 Å (e.g., equal or less than 6 Å, or even 4 Å) for the target molecule-ligand pair to be considered as having contact interaction. As another example, the contact interaction can include a change in the accessible surface area of the target molecule upon forming a complex with the ligand. In one embodiment, the interaction type can be a polar interaction, non-polar interaction, and hydrogen bond interaction. In one embodiment, the hydrogen bond interaction can include a hydrogen bond donor in the target molecule and a hydrogen bond acceptor in the ligand at the corresponding selected position. In one embodiment, the hydrogen bond interaction can include a hydrogen bond acceptor in the target molecule and a hydrogen bond donor in the ligand at the corresponding selected position. In one embodiment, the method can further include instructions to store the SIFt in a database. In one embodiment, the computer program can include instructions for generating a plurality of SIFts by the repeating the steps recited above using, e.g., the same target molecule and selected positions, but different ligands. The plurality of SIFts may then be stored in a database. In one embodiment, the computer program can further include instructions to generate a SIFt using the same target molecule and a test ligand, and to compare this SIFt with another SIFt (e.g., generated using the same target and a known ligand) or another group of SIFts (i.e., either one SIFt or a plurality of SIFts forming an interaction pattern). Various methods can be used to compare the generated SIFt with one or more other SIFts. For example, a comparison can be performed using a simple sum of matching bits (units) across the entire SIFT, or by the application of one or more similarity measures (including, e.g., Tanimoto coefficient, Euclidean distance, cosine correlation coefficient, correlation, half square Euclidean distance, and city block distance). Furthermore, a library of SIFts can be compared by, for example, first carrying out all pairwise comparisons using one of the similarity measures mentioned above and then applying hierarchical clustering to group SIFts according to the similarity. The clustering can use, for example, one or more common cluster similarity methods (including, e.g., UPGMA (Unweighted Pair-Group Method with Arithmetic mean), WPGMA (Weighted Pair-Group Method with Arithmetic mean), single linkage, complete linkage, and Ward's method).

As used herein, a target molecule generally refers a biomolecule whose functions are desired to be modulated. A target molecule contains a region (i.e., binding site) that allows it to bind to one or more ligands that satisfy the binding criteria. A target molecule can be a macromolecule such as a protein (or even a polypeptide) or a nucleic acid. A target molecule is typically a bio-macromolecule whose functions can be altered when it is bound to a molecule (i.e., ligand) that fits its binding or active site.

As used herein, a ligand refers to a molecule that binds to the binding or active site of a target molecule. A ligand is typically a smaller molecule than a target molecule and typically binds to a target molecule with high affinity (e.g., with a K_{d} of at least 1 mM). A ligand can be a natural ligand or substrate (i.e., naturally occurring in a biological system) to the target molecule, e.g., ATP to certain kinases such as p38. A ligand can also be a small molecule inhibitor, e.g., SB203580 that is a well-known inhibitor of p38.

As used herein, a naturally occurring amino acid is defined as one of the twenty amino acids naturally occurring in proteins. These naturally occurring amino acids are the L-isomers of glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine, and lysine. A so-called “unnatural” amino acids is any amino acid other than the twenty named above. Included are D-isomers of the twenty amino acids named above, D or L isomers or racemic mixtures of selenocysteine and selenomethionine, and the D or L forms (or racemic mixtures) of, e.g., nor-leucine, para-nitrophenylalanine, homophenylalanine, para-fluorophenylalanine, 3-amino-2-benzylproprionic acid, homoarginine, and the like. These unnatural amino acids may be used, e.g., in rational drug design in developing inhibitors and/or binding molecules to modulate a protein's activity.

An amino acid is a molecule having the structure where a central carbon atom (the α-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group that is linked to the α-carbon atom. For example, the side chain group of alanine is a methyl group. Any atom that is not part of a side chain group is a main chain atom, e.g., the α-carbon atom or the hydrogen that joins this carbon atom.

A positively charged amino acid is any naturally occurring or unnatural amino acid having a side chain that is positively charged under normal physiological conditions. The positively charged, naturally occurring amino acids are arginine, lysine, and histidine. A negatively charged amino acid is any naturally occurring or unnatural amino acid having a side chain that is negatively charged under normal physiological conditions. Examples of negatively charged, naturally occurring amino acids are aspartic acid and glutamic acid. A hydrophobic amino acid is any naturally occurring or unnatural amino acid that contains a hydrophobic side chain group. Examples of naturally occurring hydrophobic amino acids are alanine, leucine, isoleucine, valine, proline, phenylalanine, tryptophan, and methionine. An uncharged, hydrophilic amino acid is any naturally occurring or unnatural amino acid that is contains a hydrophilic side chain group, but is uncharged at physiological pH. Examples of naturally occurring uncharged, hydrophilic amino acids are serine, threonine, tyrosine, asparagine, glutamine, and cysteine.

As used herein, a polypeptide refers to a polymer of two or more amino acids linked via a peptide bond (i.e., amino acid residues), and occurs when the carboxyl carbon atom of the carboxylic acid group bonded to the α-carbon of one amino acid (or amino acid residue) becomes covalently bound to the amino nitrogen atom of the amino group bonded to the α-carbon of an adjacent amino acid. A protein can include one or more polypeptide subunits (e.g., DNA polymerase III, RNA polymerase II) or other components (e.g., an RNA molecule, as occurs in telomerase) will also be understood to be included within the meaning of “polypeptide” as used herein. Similarly, fragments of full-length proteins are also “polypeptides”.

The amino acid sequence of a given naturally occurring polypeptide (i.e., the polypeptide's “primary structure”) can be determined by the nucleotide sequence of the coding portion of a mRNA, which is in turn specified by genetic information, typically genomic DNA (including organelle DNA, e.g., mitochondrial or chloroplast DNA).

The secondary structure of a polypeptide refers to local regular structure of a polypeptide segment, without considering the conformations of the side chain its residues. Common secondary structure elements include α-helix and β-strand. The tertiary structure refers to the three-dimensional arrangement of all atoms in a polypeptide chain.

An amino acid residue of a polypeptide interacts with adjacent residues (e.g., residues that are adjacent in primary, secondary or tertiary structure of a polypeptide) as well as with ligands or substrates based, in part, on the type of side chain group present. For example, hydrophobic amino acids are more likely to interact with other hydrophobic amino acids or hydrophobic molecules. Similarly, hydrophilic amino acids are more likely to interact with other hydrophilic amino acids or hydrophilic molecules. These types of interactions can be identified and characterized as discussed herein based upon a residues chemical characteristics as well as its interaction with adjacent atoms or molecules.

As used herein, a nucleic acid refers to DNA and RNA, which are both linear polymers of nucleotide subunits. Each nucleotide unit contains a base, a sugar and a phosphate. In DNA, the sugar is deoxyribose, and there are four types of bases: adenine (A), thymine (T), guanine (G), and cytosine (C). In RNA, the sugar is ribose, and bases are made up of adenine (A), uracil (U), guanine (G), and cytosine (C). In either DNA and RNA, the base is linked to the sugar moiety through a beta-glycosyl linkage, and the nucleotide units are joined together through phosphodiester bonds with phosphates at O3′ and O5′ of the sugars.

The details of one or more embodiments are set forth in the accompanying drawings and the description below. Other features, objects, and advantages will be apparent from the description and drawings, and from the claims.

### DESCRIPTION OF DRAWINGS

FIG. 1 is a flow chart depicting a method of generating a SIFt.

FIG. 2A is an overlay of 100 different docking poses of SB203580 (shown in cyan stick models) in the vicinity of the target protein human p38 (PDB accession code: 1a9u). p38 is shown as ribbon model, and the shades represent different sub-regions of the 34 ligand binding site residues: R—Gly-rich loop, G—segment from -β3 to β4 (including αC), B—β5 and hinge region, M—catalytic loop, Y—Mg loop, O—activation segment. A color version of this figure can be found in Deng, Z.; Chuaqui, C.; Singh, J. “Structural Interaction Fingerprint (SIFt): A novel method for analyzing three-dimensional protein-ligand binding interaction,” J. Med. Chem, 47: 337-344 (2004).

FIG. 2B is a hierarchical clustering of the SIFts of 100 SB203580 docking poses. A color version of this figure can be found in Deng, Z. et al., J. Med. Chem, 47: 337-344 (2004). Each SIFts is represented as one line in the heat map in the middle of the figure, and only ON-bits (1) are shown as blocks. On the right side of the heat map shows the hierarchical clustering results on the fingerprints, including the dendrogram and the reorganized distance matrix. Colors (represented here as shades of gray) in the distance matrix correspond to the actual pair-wise distance between two SIFts, with dark red (e.g., cutting from top right to bottom left) being the most similar and dark blue (e.g., in the northwest and southeast corners) being the least similar. SIFts in the heat map are rearrange according to the order given by hierarchical clustering. The seven major clusters (labeled 1-7) identified from the dendrogram are marked on the left side of the SIFt heat map. The three lines of blocks above the heat map indicate the locations of the corresponding binding site residues and the bits. In the middle line (alternating shades of gray), each block represents a particular binding site residue, arranged in ascending residue numbers. Within each residue there are seven different binding bits, represented by seven smaller blocks in the third line. Also, the residues are grouped into six different regions as described in FIG. 2A, as indicated in the first line.

FIG. 2C-2I collectively are overlays of the poses within each of the seven clusters (labeled 1-7), in the same reference frame as FIG. 2A. The crystal structure of SB203580 in the 1a9u structure is also shown in each figure as stick model. Color versions of these figures can be found in Deng, Z. et al., J. Med. Chem, 47: 337-344 (2004). Among the binding site residues, only those in contact with the respective clusters are shaded, using the same scheme as in FIG. 2A.

FIG. 3A is a graph showing the PMF docking scores as a function of SIFt cluster number.

FIG. 3B is a graph showing the Consensus docking score as a function of SIFt cluster number.

FIG. 4A is a representation of ligand binding site residues of protein kinases. Shown are the murine PKA (ribbon model) and the ATP molecule (stick model) of the crystal structure latp, which was used as the reference structure for the kinase SIFt construction. Residues are grouped into five different regions, shown in shades of gray. The grouping and shading scheme are the same as in FIG. 2A. A color version of this figure can be found in Deng, Z. et al., J. Med. Chem, 47: 337-344 (2004).

FIG. 4B is a hierarchical clustering of SIFts of 89 protein kinase crystal structures. On the right are the dendrogram and the corresponding reorganized distance matrix map. SIFts are reorganized according to the order given by the dendrogram. Six different regions are labeled above the SIFt heat map. Three major clusters (1 -3) are labeled on the left side of the heat map. A color version of this figure can be found in Deng, Z. et al., J. Med. Chem, 47: 337-344 (2004).

FIG. 4C is a comparison of the structures of the three different binding modes from FIG. 4B. Three representatives are shown for each cluster.

FIGS. 5A and 5B are graphs showing the comparison of database enrichment using SIFt with ChemScore (FIG. 5A) and PMF score (FIG. 5B). Sixteen known p38 inhibitors were diluted in 1,000 diverse compounds. For each compound, 30 different docking poses were retained and their respective ChemScores and Tanimoto coefficients (compared with the crystal structure 1a9u) were calculated. The best Tanimoto coefficient among the 30 docking poses of a compound is plotted against the best ChemScore or PMF score of the same molecule. The dark dots in the figures represents the 16 known inhibitors, and the lighter dots represent the 1,000 random compounds. The dotted lines indicate the corresponding cut-off scores used to filter the docking poses in order to recover 14 out of 16 (87.5%) known inhibitor. Color versions of these figures can be found in Deng, Z. et al., J. Med. Chem, 47: 337-344 (2004).

FIG. 6 is a schematic example of an embodiment (i.e., bit-string) of the method of FIG. 1.

FIG. 7 A is a schematic diagram depicting the decomposition of a molecule into a core and variable groups.

FIG. 7B is a hierarchical clustering of the SIFts of 100 docking poses. The SIFts are constructed to represent different R-groups and the core of the molecule. Each selected position of the target molecule is made up of four binary bits, representing core, R1, R2, R3, and R4, respectively. Each SIFts is shown as one line in the heat map in the left of the figure, and only ON-bits are shown. The shades (colors) of the heat map blocks indicate different R-groups: red—core, blue—R1, yellow—R2, green—R3. On the right side of the figure shows the hierarchical clustering results on the fingerprints, including the dendrogram and the reorganized distance matrix. SIFts in the heat map are reorganized according to the order given by the hierarchical clustering. The shaded (colored) bar on top of the SIFt heat map represents five corresponding kinase structural sub-regions in the fingerprints. These sub-regions, each shaded (colored) differently, include the Gly-rich loop (G-loop), the region spanning from β3 to β4 (β3 to β4), β5 and the hinge region, catalytic loop and magnesium loop.

FIG. 7C and 7D show the structures of the poses in cluster 1 (7C) and cluster 2 (7D), respectively, as identified by the hierarchical clustering of their R-SIFts (FIG. 7B), in the context of the p38 crystal structure (1a9u). The poses are shown in gray, and the co-crystal structure of SB203580 is shaded according to atom types. The five kinase sub-regions that are in contact with the poses within the group are shaded using the same shading scheme as described in FIG. 2B and FIG. 7B.

FIG. 8 is a hierarchical clustering of the SIFts of the 100 docking poses. Here the SIFt patterns contain 7 bits per selected position, each representing one of the seven chemical features of the molecule: red—hydrogen bond acceptor (HBA), blue—hydrogen bond donor (HBD), yellow—hydrophobic (HPH), green—polar (POL), cyan—negatively charged (NEG), orange—positively charged (POS), black—aromatic ring (AROM). The hierarchical clustering is based on the new SIFt patterns incorporating the chemical features of the molecules.

FIG. 9A is an interaction profile generated from the SIFt patterns of four p38 crystal structures—1a9u, 1b16, 1b17, and lbmk. The X-axis are the p38 residue numbers of the interaction bits; the Y-axis represents the conservation scores of the interaction bits.

FIG. 9B shows the p38 inhibitor database enrichment performance using the SIFt-based approach. A library comprised of 16 known p38 inhibitors and 1000 random compounds were docked onto p38 target molecule and enriched using the SIFt-based Z score ranking method. The X-axis is the percentage of the whole library collected, and the Y-axis is the percentage of active compounds harvested. For comparison, the enrichment performances by two conventional scoring functions (ChemScore and PMF Score) are also shown.

### DETAILED DESCRIPTION

As used herein and in the appended claims, the singular forms “a,” “and,” and “the” include plural referents unless the context clearly dictates otherwise. Thus, for example, reference to “a protein” includes a plurality of proteins and reference to “the polypeptide” generally includes reference to one or more polypeptides and equivalents thereof known to those skilled in the art, and so forth.

Unless defined otherwise, all technical and scientific terms used herein have the same meaning as commonly understood to one of ordinary skill in the art. Although any methods, devices and materials similar or equivalent to those described herein may be used, the typical methods, devices and materials are now described.

All publications mentioned herein are incorporated herein by reference in full for the purpose of describing and disclosing the databases, proteins, and methodologies described in the publications that might be used in connection with the presently described techniques. The publications discussed above and throughout the text are provided solely for their disclosure prior to the filing date of the present application. Nothing herein is to be construed as an admission that the inventors are not entitled to antedate such disclosure by virtue of prior invention.

Techniques are provided for a simple and robust method for representing and analyzing three-dimensional target molecule-ligand interactions. This method generates a structural interaction fingerprint (SIFt)—a representation of the interactions in the three-dimensional binary complexes, i.e., target molecule-ligand (e.g., protein-ligand or nucleic acid-ligand) complexes. The representation is in the form of an information string (e.g., a binary bit string) containing a plurality of information blocks; each of which, in turn, contains a plurality of information units. Before one constructs a SIFt, one has to select the binary (target molecule-ligand) complexes.

A. Construction and Analysis of SIFts

I. Selection of Three-Dimensional Binary Complex Structures

The SIFt-based method employs a set of three-dimensional binary structures (e.g., the molecular docking results) to generate a set of SIFts. The set of structures can be obtained from different poses of a selected pair of target molecule (e.g., a protein such as a kinase) and ligand (e.g., a natural ligand or an inhibitor). See, e.g., Example 1 wherein the set of structures was obtained from 100 of different poses of a pyridinyl imidazole inhibitor docking onto a single protein kinase p38 structure. In another aspect the set of structures can be obtained from structural data (e.g., docking results) of a number of different ligands interacting with a single target molecule. See, e.g., Example 2 wherein the set of structures was obtained from docking a group of different small molecules (a library of 1,016 small molecules) onto the same target molecule (a protein kinase p38 structure). In a further aspect, the set of structures can be obtained from different target molecules and different ligands (see, e.g., Example 3 wherein both the target molecules (protein kinases) and ligands are different). Using different target molecules requires additional structural and sequence alignment steps, which will be further discussed below. Once a set of structures has been obtained, one can proceed to construct SIFts.

II. Construction of a SIFt

(i) Identification of the Selected Positions of a Target Molecule

The next step involves selection of a set of positions (“selected positions”) on the target molecule of each of the structures where each of these selected positions is commonly involved in interactions (e.g., non-covalent interaction) between the target molecule and the ligand. These positions serve as reference points covering all of the interactions in the target molecule-ligand complex, and are then used as the common reference frame for constructing SIFts.

But how does one determine the location of the interactions between the target molecule and the ligand? The selected positions are defined as regions of the target molecule that are in contact with the ligand. Different methods have been developed to determine whether contacts have been made between the target molecule and the ligand in the context of a particular interaction. Below is a description of two exemplary methods.

For example, the program AREAIMOL of the CCP4 suites (which refers to “Collaborative Computational Project, Number 4.” See the CCP4 suite: programs for protein crystallography. Acta Cryst., D50, 760-763, 1994; and Lee et al., J. Mol. Biol. 55:379-400, 1971) can be used to identify the target molecule atoms that are involved in the non-covalent intermolecular interactions with the ligand. AREAIMOL evaluates the covalent accessible area by allowing a probe sphere of 1.4 Å rolling over the Van der Waals surface of the target molecule and the target molecule-ligand complex. Note that solvent molecules can be excluded for the sake of simplicity, although in theory well-ordered solvent molecules can be included and treated in the same way as target molecule atoms. For protein target molecules, if non-hydrogen atoms show that solvent accessibility decreases upon ligand binding and these atoms are also within 4.5 Å of any of the non-hydrogen atoms of the ligand, the residues corresponding to these atoms are identified as selected positions (or ligand binding atoms). The determination of selected positions in nucleic acid can be done in a similar manner.

As to hydrogen bonding interaction between the target molecule and the ligand, one can employ programs such as HBPLUS. See McDonald et al., J. Mol. Biol. 238:777-793, 1994. HBPLUS calculates and list all possible hydrogen bond donor and acceptor pairs in the complex.

For a set of structures using the same target molecule, after all the ligand binding atoms and their respective residues or bases have been identified, these ligand binding positions are computed and defined as the “selected positions” of the target molecule. As mentioned above, different target molecules can be used. In such circumstances, additional structural and sequence alignment steps are required to convert different but related target molecules into a standard residue numbering system so that a common framework can be employed for constructing the SIFts (see, e.g., Example 3).

(ii) Determination and Calculation of Interaction Types

After identification of the selected positions (i.e., regions of the target molecule where intermolecular interactions take place), one has to determine and calculate the types of interactions present at these positions. In one embodiment, the target molecule can be a polypeptide or a protein and seven interaction types can be employed based on the AREAIMOL and HBPLUS results. The presence or absence of the interaction types can be calculated at each selected position based on the following inquiries: 1) whether or not it is in contact with the ligand; 2) whether or not any peptide backbone atom is involved in the contact; 3) whether or not any side-chain atom is involved in the binding; 4) whether or not polar interaction is involved; 5) whether or not non-polar interaction is involved; 6) whether or not this residue provides hydrogen bond acceptor(s); and 7) whether or not it provides hydrogen-bond donor(s). The answer to each inquiry constitutes an information unit (in this embodiment, a bit) that corresponds to a particular selected position. By joining the information units together, an information block is formed (in this embodiment, a seven-bit-long block). The entire SIFt can then be constructed by sequentially concatenating the information blocks of each of the selected positions together, according to ascendant position number (e.g., residue number) order.

The SiFts resulting from a set of structures are therefore of the same length, and each information unit (e.g., bit) in the fingerprint represents the strength or the presence/absence of a particular interaction type at a particular selected position. As a result, the SIFts are directly comparable. Once SiFts are generated from a set of structures, one can perform analyses of the SIFts to obtain valuable interaction patterns and information (e.g., the degree of binding conservation among the target molecule-ligand pairs).

The interaction types can be classified in a number of ways. For example, the interaction types can be fragment constants descriptors (e.g., hydrophobicity, hydrogen bond acceptor, hydrogen bond donor), electronic descriptors (e.g., charge, partial positive surface area, partial negative surface area, dipole movement, atomic polarizability), topological descriptors (e.g., Wiener index, Zagreb index, Hosoya index), molecular flexibility indices, spatial descriptors (e.g., shadow indices, molecular surface area, density, principal moment of inertia, molecular volume), structural descriptors (number of chiral centers, molecular weight, number of rotatable bonds), or thermodynamic descriptors (e.g., partition coefficient, desolvation free energies for water and octanol, pKa).

Hydrophobicity is a measure of the thermodynamics of the partitioning of a molecule or part of a molecule between water and a non-aqueous phase (e.g., an organic solvent), in particular, the free energy change (ΔG⁰_{transfer}) associated with transferring a molecule or part of the molecule from a non-aqueous phase to water. In one popular definition (CATALYST™, Accelrys Inc., San Diego, Calif. 92121, USA), a contiguous set of atoms are defined as hydrophobic if they are not adjacent to any concentrations of charge (charged atoms or electronegative atoms), in a conformation such that the atoms have surface accessibility, including phenyl, cycloalkyl, isopropyl, and methyl.

III. Analysis of SIFts

(i) Measurement of Similarity of SIFts

As discussed above, each SiFt represents the interaction profile between a target molecule and a ligand. It follows that similar SIFts reflect similar interaction patterns among the target molecule-ligand pairs.

Different methods can be employed to measure similarity between SIFts. For example, one can use Tanimoto coefficient (Tc, see Willet, Chem. Inf. Comput. Sci. 38:983-996, 1998), which reflects the quantitative measurement of the similarity. Using the bit-string embodiment described above, Tc between bit-strings A and B is defined as

$$Tc ( A , B ) = \frac{\vert A ⋂ B \vert}{\vert A ⋃ B \vert} ,$$

where |A ∩B| is the number of ON-bits common in both A and B and |A ∪B| is the number of ON-bits present in either A or B.

(ii) Classification of SIFts Based on Similarity

Based on the similarity measurements, one can classify similar SIFts displaying similar interaction patterns for further analysis, using methods such as hierarchical clustering.

From the clustering results, structures can be clustered into groups having similar binding modes.

To analyze and compare the interaction patterns within a group or between groups, an interaction profile can be generated by quantifying the degree of similarity of each information unit at each selected position within the SIFts. One example is to calculate an interaction conservation score for each information unit (e.g., bit) among each group. This score represents the percentage of SIFts that is ON (i.e., occurrence or presence of the interaction type) at this particular selected position. The higher the score, the more conserved this interaction type is within this group. Variations in the conservation scores between two groups reveal the differences of their interaction patterns.

B. A High-Level view of the SIFt-Based Method

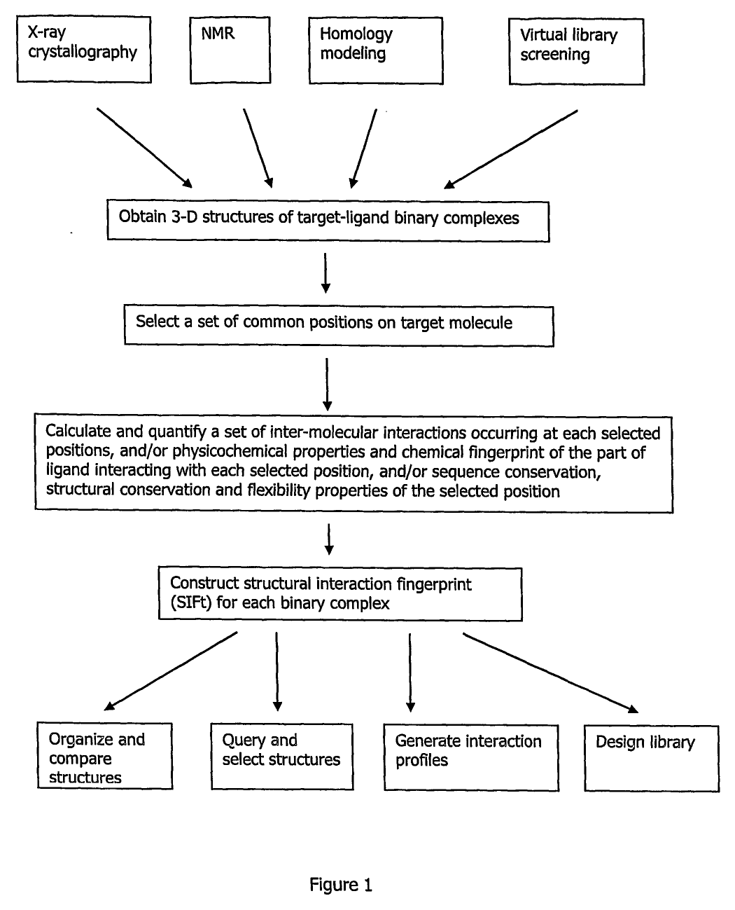

FIG. 1 shows a high-level view of an exemplary method for generating a SIFt. The method utilizes entries contained in structural databases containing data from various sources, e.g., X-ray crystallography, NMR, protein modeling, and/or protein/ligand interaction simulations (100). At block 200, three-dimensional data/structures of one or more complexes are retrieved from a database. Using any of a variety of computational methods well known to those in the art, a set of selected positions (e.g., amino acid residues or bases) that interact with a putative ligand or binding molecule are selected at block 300.

Once a three dimensional structure has been derived and selected positions (e.g., binding site residues) identified, a plurality of intermolecular interaction types occurring at each selected position is determined and measured at block 400, using any computational methods well known in the art. These interaction types can also include chemical and physical properties of the part of a ligand interacting with each selected position, and sequence conservation, structural conservation and flexibility properties of each selected position.

At block 500, a SIFt for each target molecule-ligand complex structure is generated. The SIFt includes a numeric (e.g., binary) code representation of each interaction type determined/measured for each of the selected positions of the target molecule.

At block 600, the SIFt containing information regarding characteristic of the interaction types at each selected position is stored within a database for subsequent retrieval and analysis. Alternatively, the SIFt can be used to query a database (block 650), generate an interaction profile comprising possible alternative ligands that fit the SIFt (block 625), and/or define a structure based upon the type of SIFt obtained (block 675).

In one embodiment, a primary amino acid sequence of a polypeptide target molecule that is encoded by a selected genetic sequence is determined, and a three-dimensional structure is generated by homology modeling techniques. This aspect is generally represented in FIG. 1 as block(s) 100. As mentioned above, a three-dimensional model of a particular target molecule may be predicted computationally or determined in whole or in part based on experimental information. For example, x-ray crystallographic information may be used to identify a protein structure and provide information for constructing a three-dimensional model of the protein target molecule.

In one embodiment, a ligand's three-dimensional structure is also obtained by similar techniques (e.g., modeling techniques and/or experimental crystallization techniques). For example, many protein molecules are co-crystallized with substrates and/or ligands. The three-dimensional ligand binding structure can then be modeled using programs that demonstrate interactions with a putative protein target molecule or binding domain thereof. Thus, one of skill in the art utilizing the 3D-protein structure and/or the 3D-ligand structure can obtain interaction data for the molecules being characterized. The ligand molecule may be any of a number of different types of compositions such as organic molecules, inorganic molecules, ions, proteins, protein fragments, nucleotides, RNA, DNA or other molecules representative of substrates, ligands, co-factors, and the like. In one embodiment, the ligand is obtained from a library of molecules.

Upon formation of the 3D complex structure, the interaction of the target molecule with a ligand is computed. Positions (e.g., amino acid residues) that play a role in the interaction with the ligand are selected. This is generally represented by block 300 of FIG. 1. Particular atoms in the ligand can be identified as interacting with particular amino acid residues or bases of the target molecule. The criteria for determining an interaction (e.g., distance (e.g., in angstroms) between various atoms) can be adjusted using techniques in the modeling programs as mentioned above or by techniques known to those skilled in the art.

The target molecule-ligand interactions that are modeled result in the identification of certain selected positions (e.g., amino acid residues or bases) as well as the nature of interaction types between the ligand and the target molecule. The interaction types between a ligand and a particular selected position will depend upon the chemical-physical characteristics of the selected position in the target molecule as well as the nature of atoms or groups of atoms present in the ligand. For example, one of skill in the art will recognize that various equilibrium binding constants or binding energy values will be determinative in the type of interactions that will occur. This process is represented in FIG. 1 by block 400.

The selected positions that play a role in interacting with the ligand as well as the interaction types that occur with each selected position are then used to generate a SIFt (see, e.g., block 500 of FIG. 1). This SIFt can be represented by a series of numerical values (e.g., binary numbers) corresponding to each selected position and each interaction type. The selected position and interaction type form a SIFt that can be used to compare or distinguish the target molecule (or a family of target molecules) from other target molecules. Using the SIFt as a tool for comparison, target molecules (e.g., proteins or polypeptides) may be structurally or functionally associated when they share commonalities in the SIFts. This latter process is represented in FIG. 1 by block 675. For example, by aligning the SIFts of two protein target molecules, a functional relationship can be determined based upon the degree of alignment (e.g., homology) between the two information strings or SIFts. Various statistical measurements and limits can be placed upon the alignment to discriminate between random and related alignments. Accordingly, a powerful tool is provided to associate target molecules in a manner that does not rely on sequence or homology matching/comparisons alone, and to allow for the association of otherwise dissimilar target molecules that can be functionally related by their SIFts.

In certain embodiments, the SIFt fingerprint records the presence or absence of an interaction with a protein. The information unit containing this information can be simple to indicate whether a residue is involved in a particular interaction or not. In other embodiments, the SIFt can also include other chemical information about the ligand. In one example, a SIFt can include an information unit that contains information about a combinatorial library, which can include a core and variable group (in some examples, two, three or more R groups). Specifically, a small molecule library can be converted into a core and variable groups, a SIFt pattern can be created for each library member, information units can be turned on or off at each of the selected positions based on the nature of the contact between the core and variable groups with the protein target. In another example, a SIFt can include an information unit that contains chemical feature information. For example, a series of chemical features can be mapped onto the ligand molecule. Each residue can be represented by an information block of a series of information units, each of which can be turned on or off depending on whether this residue is interacting with a particular chemical feature on the ligand. Examples of suitable chemical features include hydrophobic, hydrogen bond donor, hydrogen bond acceptor, negatively charged, positively charged, etc. In another example, a computed or experimentally determined property can be included in a SIFt. Information blocks that includes these properties can be used to identify chemical groups that are associated with specific residues of the protein.

C. Embodiments and Applications

As discussed above, one embodiment involves the use of a seven-bit information block (e.g., contact, main-chain atom group, side-chain atom group, polar, non-polar, hydrogen bond donor, hydrogen bond receptor) to represent the interaction pattern of each selected position of the target molecules (e.g., binding site residue of a protein target molecule). In such an embodiment, the interaction pattern represents the binding modes formed from seven different interaction types. Although such implementation has been shown to be able to successfully organize, analyze and mine a large structural library in a meaningful way, a 7-bit-long binary string obviously does not represent all the intermolecular interactions occurring at a particular selected position. The richness of information can be improved by incorporating more bits representing other interaction types. For example, one can focus on functional groups instead of the entire residue as the basic unit, or take solvent molecules into consideration, or substitute the BOOLEAN bits with ordinal or continuous data that reflect the strength and energetics of the interaction types. Such enriched SIFt provides a “higher-resolution” picture of the target molecule-ligand binary complex. In situations where computational speed is a critical issue, “lower-resolution” SIFts using fewer information units may be used. Accordingly, the information units for a particular selected position (i.e., the size of the information block) may range from 1-50 units or more. Simpler SIFts can be constructed using shorter time at the expense of richness of information. One skilled in the art can design, select, and identify the number of information units (and thus the size of the information block) for a particular selected position based upon the details and speed desired. For example, shorter information strings (containing, e.g., 2-3 information units per information block) may be useful during the initial screening of a huge virtual library. On the other hand, longer information strings (and hence longer SIFts) provide more information at the expense of quick performance and are more useful for detailed structural analysis such as comparing groups of closely related structures. Choosing the right size of SIFt is a matter of finding a proper balance between these two competing considerations, with that balance dictated by the needs of a given situation. Another variable is the relative weight given to each interaction type. In one embodiment, information units reflecting each interaction type can contribute equally to the total similarity score. It is also possible to tailor them in a different way by focusing on one or more particular interaction types, while down-playing other kinds of interactions.

One advantageous feature of the SIFt-based method is that it is generic. Although it works well for the protein target molecule and small molecule ligand system, the method can also work for other systems as well, including protein-protein, nucleic acid-ligand, nucleic acid-protein/polypeptide systems, and the like. Indeed, the methods and systems are applicable to amino acid sequences, as well as nucleotide sequences. For example, the methods can be applied to a nucleotide sequence or an amino acid sequence which corresponds to the nucleotide sequence in question. If the coding sequence is not known, translation from the nucleotide sequence to the amino acid sequence may be performed in all frames of the nucleotide sequence. Programs that can translate a nucleotide sequence are known in the art.

In one embodiment, the method can start by identifying a primary amino acid sequence of a protein. A number of source databases are available, as described below, that contain nucleotide sequences and/or deduced amino acid sequences for use with this step.

The primary direct experimental methods for determining the structure of proteins involved in particular interactions are X-ray crystallography, relying on the interaction of electron clouds with X-rays; and liquid nuclear magnetic resonance (NMR), relying on correlations between polarized nuclear spins interacting via indirect dipole-dipole interactions. X-ray methods provide information on the location of every heavy atom in a crystal of interest, accurate to 0.5-2.0 Å (1 Å=10-8 cm).

A number of databases are available that contain 3D protein structures and/or structures showing 3D protein-ligand interactions. For example, protein-protein interaction databases include the Biomolecular Interaction Network Database (BIND), which is a database designed to store full descriptions of interactions, molecular complexes and pathways; Database of Interacting Proteins (DIP), which catalogs experimentally determined interactions between proteins; an Object Oriented Database for Protein-Protein Interactions (INTERACT); and Pronet Online, which provides protein-protein interaction data and is maintained by Myriad Genetics. Other structural databases include Cambridge Crystallographic Data Centre; CATH—Protein Structure Classification; SCOP (Structural Classification of Proteins), based upon 3D fold classifications; PARTS LIST, which dynamically performs comparative fold surveys and is built on top of SCOP's fold classification and acts as an accompanying annotation; PDB (Protein Data Bank), which is an international repository for the processing and distribution of 3D macromolecular structure data primarily determined experimentally by X-ray crystallography and NMR; PRESAGE, a database for structural genomics; Structural Biology Software Database, a software database maintained by University of Illinois; BiMSSECOST, a conformational database for amino acid residues in proteins; BioMagResBank, a repository for data on proteins, peptides, and nucleic acids from NMR spectroscopy; SWISS-3DIMAGE 3D, which contains images of proteins and other biological macromolecules; SWISS-MODEL, a repository of structures generated by protein modeling; and the Cambridge Structural Database (CSD) of the Cambridge Crystallographic Data Center (CCDC). Other sources of primary amino acid sequence, modeled 3D structures and other crystallographical data will be apparent to those of skill in the art.

The various techniques, methods, and aspects described above can be implemented in part or in whole using computer-based systems and methods. Additionally, computer-based systems and methods can be used to augment or enhance the functionality described above, increase the speed at which the functions can be performed, and provide additional features and aspects as a part of or in addition to those described elsewhere in this document. Various computer-based systems, methods and implementations in accordance with the above-described technology are presented below.

In one implementation, a general-purpose computer may have an internal or external memory for storing data and programs such as an operating system (e.g., DOS, Windows 2000™, Windows XP™, Windows NT™, OS/2, UNIX or Linux) and one or more application programs. Examples of application programs include computer programs implementing the techniques described herein, authoring applications (e.g., word processing programs, database programs, spreadsheet programs, or graphics programs) capable of generating documents or other electronic content; client applications (e.g., an Internet Service Provider (ISP) client, an e-mail client, or an instant messaging (IM client) capable of communicating with other computer users, accessing various computer resources, and viewing, creating, or otherwise manipulating electronic content; and browser applications (e.g., Microsoft's Internet Explorer) capable of rendering standard Internet content and other content formatted according to standard protocols such as the Hypertext Transfer Protocol (HTTP).

One or more of the application programs may be installed on the internal or external storage of the general-purpose computer. Alternatively, in another implementation, application programs may be externally stored in and/or performed by one or more device(s) external to the general-purpose computer.

The general-purpose computer includes a central processing unit (CPU) for executing instructions in response to commands, and a communication device for sending and receiving data. One example of the communication device is a modem. Other examples include a transceiver, a communication card, a satellite dish, an antenna, a network adapter, or some other mechanism capable of transmitting and receiving data over a communications link through a wired or wireless data pathway.

The general-purpose computer may include an input/output interface that enables wired or wireless connection to various peripheral devices. Examples of peripheral devices include, but are not limited to, a mouse, a mobile phone, a personal digital assistant (PDA), a keyboard, a display monitor with or without a touch screen input, and an audiovisual input device. In another implementation, the peripheral devices may themselves include the functionality of the general-purpose computer. For example, the mobile phone or the PDA may include computing and networking capabilities and function as a general purpose computer by accessing the delivery network and communicating with other computer systems. Examples of a delivery network include the Internet, the World Wide Web, WANs, LANs, analog or digital wired and wireless telephone networks (e.g., Public Switched Telephone Network(PSTN), Integrated Services Digital Network (ISDN), and Digital Subscriber Line (xDSL)), radio, television, cable, or satellite systems, and other delivery mechanisms for carrying data. A communications link may include communication pathways that enable communications through one or more delivery networks.

In one implementation, a processor-based system (e.g., a general-purpose computer) can include a main memory, preferably random access memory (RAM), and can also include a secondary memory. The secondary memory can include, for example, a hard disk drive and/or a removable storage drive, representing a floppy disk drive, a magnetic tape drive, an optical disk drive, etc. The removable storage drive reads from and/or writes to a removable storage medium. A removable storage medium can include a floppy disk, magnetic tape, optical disk, etc., which can be removed from the storage drive used to perform read and write operations. As will be appreciated, the removable storage medium can include computer software and/or data.

In alternative embodiments, the secondary memory may include other similar means for allowing computer programs or other instructions to be loaded into a computer system. Such means can include, for example, a removable storage unit and an interface. Examples of such can include a program cartridge and cartridge interface (such as the found in video game devices), a removable memory chip (such as an EPROM or PROM) and associated socket, and other removable storage units and interfaces, which allow software and data to be transferred from the removable storage unit to the computer system.

In one embodiment, the computer system can also include a communications interface that allows software and data to be transferred between computer system and external devices. Examples of communications interfaces can include a modem, a network interface (such as, for example, an Ethernet card), a communications port, and a PCMCIA slot and card. Software and data transferred via a communications interface are in the form of signals, which can be electronic, electromagnetic, optical or other signals capable of being received by a communications interface. These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other suitable communications channels.

In this document, the terms “computer program medium” and “computer usable medium” are generally used to refer to media such as a removable storage device, a disk capable of installation in a disk drive, and signals on a channel. These computer program products provide software or program instructions to a computer system.

Computer programs (also called computer control logic) are stored in the main memory and/or secondary memory. Computer programs can also be received via a communications interface. Such computer programs, when executed, enable the computer system to perform the features as discussed herein. In particular, the computer programs, when executed, enable the processor to perform the described techniques. Accordingly, such computer programs represent controllers of the computer system.

In an embodiment where the elements are implemented using software, the software may be stored in, or transmitted via, a computer program product and loaded into a computer system using, for example, a removable storage drive, hard drive or communications interface. The control logic (software), when executed by the processor, causes the processor to perform the functions of the techniques described herein.

In another embodiment, the elements are implemented primarily in hardware using, for example, hardware components such as PAL (Programmable Array Logic) devices, application specific integrated circuits (ASICs), or other suitable hardware components. Implementation of a hardware state machine so as to perform the functions described herein will be apparent to a person skilled in the relevant art(s). In yet another embodiment, elements are implanted using a combination of both hardware and software.

In another embodiment, the computer-based methods can be accessed or implemented over the World Wide Web by providing access via a Web Page to the methods described herein. Accordingly, the Web Page is identified by a Universal Resource Locator (URL). The URL denotes both the server and the particular file or page on the server. In this embodiment, it is envisioned that a client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol (HTTP)). The selected page is then displayed to the user on the client's display screen. The client may then cause the server containing a computer program to launch an application to, for example, perform an analysis according to the described techniques. In another implementation, the server may download an application to be run on the client to perform an analysis according to the described techniques.

The described techniques open up the possibility of using an informatics approach in three-dimensional structure analysis and structure-based drug discovery. One application is in the area of virtual chemical library screening process. As discussed herein, SIFt can serve as a post-docking molecular organizer and filter. Docking poses can be organized based on their overall interaction patterns or binding modes. Furthermore, any previously acquired knowledge can be applied as structural constraints to filter out unwanted poses, giving a smaller and better pool of lead compounds. Compared to pharmacophore-based filters, the SIFt-based method is far more generic, flexible and easy to apply. In combination with other pre-existing approaches such as empirical docking scores, the SIFt-based method can weed out more false-positive compounds with undesirable properties, leaving a smaller but better pool of lead compounds, and thus significantly improve the hit rate.

In addition, the SIFt-based approach can be applied in designing, refining and pruning target-focused chemical libraries. As shown in example 4, different embodiments of SIFt (e.g., R-SIFt) can be very effective tools for discriminating compounds with different binding modes. With R-SIFt, one can easily distinguish compounds that bind to the target molecule with desirable binding mode(s) (“good molecules”) and others that do not (“bad molecules”). Based on this compound classification result, we can then generate prediction models (e.g., decision tree, neural network, support-vector machine) to predict the “good” and the “bad” compounds using their chemical properties as predictors. Such prediction models can be applied in the early stage of virtual library screening to filter out undesirable compounds in order to generate a smaller, target-specific pool of compounds.

Besides processing the virtual structures generated during chemical library screening, the SIFt-based method can be used to analyze experimentally determined structures. Furthermore, the methods are not limited to structures involving one particular target molecule; the method is generic enough to work for structures of a family of target molecules (e.g., the kinase family). The prerequisite is that these target molecules are structurally related, so that a common framework of the ligand-binding site can be constructed. By using this method, distinct sub-groups of target molecule-ligand (e.g., enzyme-inhibitor) complex structures, each of which represents a distinct overall interaction pattern, can be identified. The identified sub-groups of these target molecule-ligand complexes can also be classified according to other grouping criteria, such as grouping by different target molecule, by different types of ligands, or by different conformations. Quantitative comparisons of these clusters would reveal interaction patterns specific for a particular group and thus could provide structural insight into the mechanism of binding activity and selectivity. In addition, the SIFt-based interaction profile can capture the common features among a group of ligand-target molecule structures. It can be used to compare different groups of structures, and to correlate the differences or commonality in their SIFt profiles to their activities.

In sum, the methods of characterization and generation of information strings representing SIFts provided by the described techniques are an improvement over conventional characterization methodologies that typically rely on sequence-based comparisons. The SIFt facilitates and integrates several desirable functionalities including structural data visualization, organization, analysis, and mining together, making it an powerful tool for analyzing and profiling three-dimensional binding interactions. As mentioned above, a particular useful feature of this method is that it compares and reveals associations (e.g., binding similarities) between dissimilar target molecules (e.g., proteins that may have functional or behavioral analogies but are not obvious due to differences in the protein sequence).

The described techniques (including SIFt-based methods, computer implementations, systems, and databases) disclosed herein translate three-dimensional intermolecular interactions into simple, linear information strings, thereby making it possible to efficiently analyze large libraries of structures using mathematics and informatics methods described herein. Although conceptually simple, the described techniques provide a novel method of visualizing, organizing, analyzing, and mining 3D structural information. The SIFt method organizes target molecule-ligand complex structures into groups based on their interaction patterns. Intermolecular interactions between target molecules and ligands are visualized and can be easily comprehended using the heat-map of the SIFts for data visualization. Specifically, each line representing one fingerprint (or SIFt), and each bit in the SIFt colored or shaded according to its value. Using the described techniques, conserved/unconserved interactions within or among different sub-groups of structures (data analysis) can be compared and quantified. In addition, by representing the target molecule-ligand complex structures using SIFts, a query can be performed based upon structural interactions to select complexes (or ligands) that satisfy predefined criteria (e.g., a certain interaction pattern or binding mode, or even a particular interaction type occurring at a selected position), in a way similar to querying a database (data mining).

### EXAMPLES

The following examples are provided to illustrate the practice of the described techniques, and in no way limit the scope of the claims.

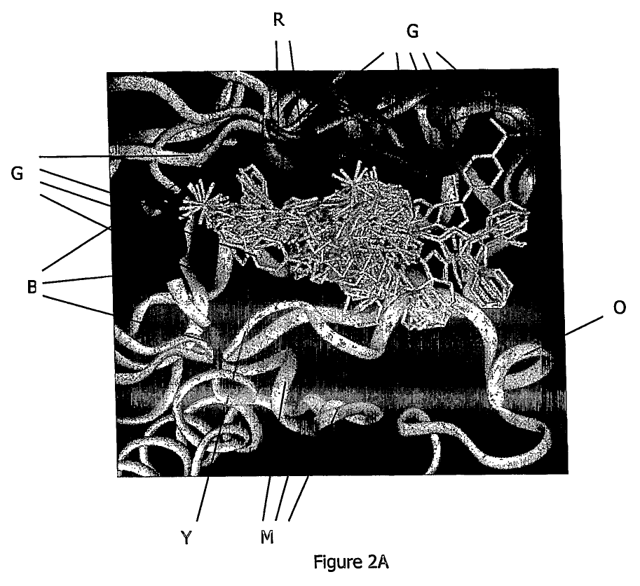

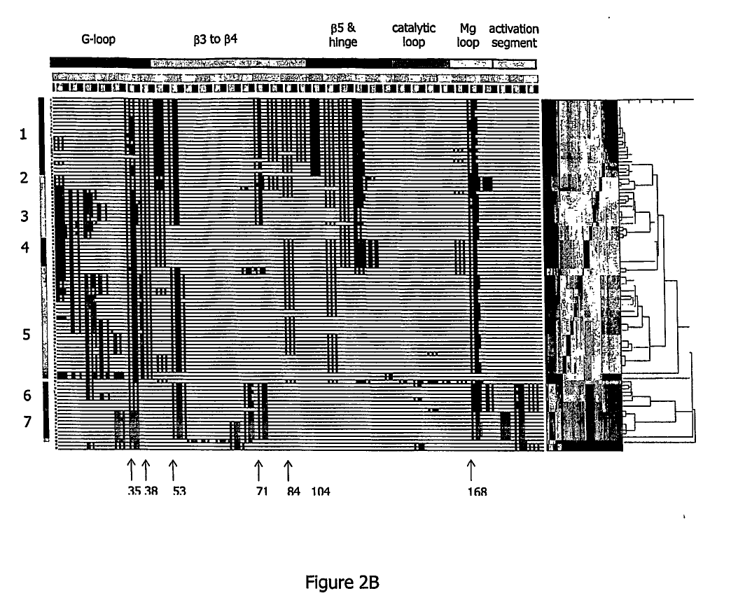

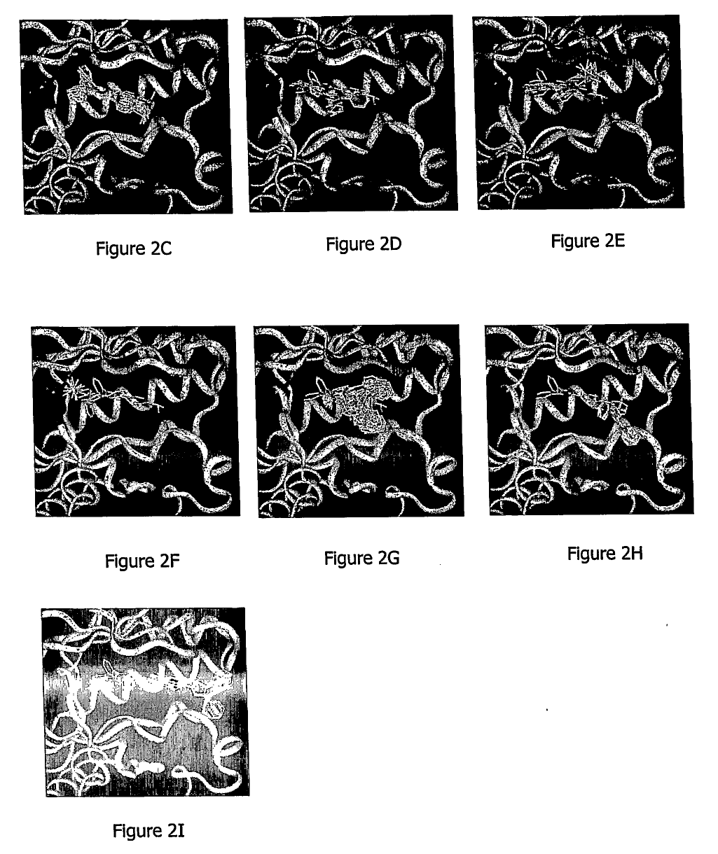

Color versions of FIGS. 2A-5B can be found in Deng, Z.; Chuaqui, C.; Singh, J. “Structural Interaction Fingerprint (SIFt): A novel method for analyzing three-dimensional protein-ligand binding interaction,” J. Med. Chem, 47: 337-344 (2004).

### Examples 1 -3

In Example 1, a set of molecular docking results was generated employing the crystal structure of p38 in complex with a pyridinyl imidazole inhibitor SB203580 (PDB accession code: 1a9u). See, e.g., Wang et al. Structure, 1998, 6(9), 1117-1128. The docking program FlexX (see Rarey et al. J Mol Biol, 1996, 261, 470-489) in Sybyl (version 6.8, Tripos, Inc., St. Louis, Mo.) was used to dock SB203580 onto the crystal structure of p38. In this single ligand study, 100 poses of SB203580 generated by FlexX were retained for subsequent analyses. The ligand binding site was defined using a cutoff radius of 12 Å from the SB203580 ligand (i.e., the conformation in the crystal structure) combined with a core sub-pocket cutoff distance of 4 Å. The FlexX scoring function was used for scoring the docking. For each ligand being studied, ChemScore, Gscore, PMF Score, Dscore, and Consensus Score were evaluated using the Cscore utility in Sybyl. For references of the just-mentioned applications, see, e.g., Eldridge et al. J Comput.-Aided Mol Des. 1997, 11, 425-445; Jones et al. J. Mol. Biol. 1997, 267, 727-748; Muegge et al. J Med. Chem., 1999, 42(5), 791-804; Gohlke et al. J Mol. Biol., 2000, 295, 337-356; and Charifson et al. J. Med. Cliem., 1999, 42(25), 5100-5109. FIG. 2A shows the 100 poses generated in this experiment, which adopted different orientations and positions in the ATP binding site of the kinase.

In Example 2, the experiment described was designed to evaluate the database enrichment potential of SIFt by docking a diverse set of compounds spiked with known actives onto the same target protein structure. To this end, 16 known p38 inhibitors were combined with 1,000 small molecules with diverse chemical structures compiled internally. These inhibitors were pyridinylimidazoles and analogs, covering the majority of the p38 inhibitor families reported thus far, as previously discussed by Adams and Lee (see Adams and Lee. Current Opinion Drug Discovery & Development. 1999, 2, 96-109). These 1,016 compounds were docked onto the p38 structure (1a9u) using FlexX distributed across 50 dual processor nodes of a Linux computing farm. For each ligand, 30 different poses generated from the docking experiment were retained, generating a library of 30,480 (30×1,016) docked ligand structures for subsequent interaction fingerprints analysis. The performance of database enrichment was measured by the enrichment factor (EF), calculated based on the ability of recovering 14 out of 16 (87.5%) known inhibitors. For reference, see, e.g., Pearlman et al. J Med. Chem. 2001, 44, 502-511. In both docking experiments, three-dimensional conformers of the ligands were generated using OMEGA (OpenEye Scientific Software, Inc., Santa Fe, N.Mex.).

In Example 3, the SIFt-based method was also used to analyze a family of experimentally determined structures. Specifically, a panel of 89 X-ray crystal structures of protein kinase-ligand complexes was selected from the PDB. The selection criteria included: 1) the structures must contain ligands (either ATP, GTP or other inhibitors) present in their ATP-binding pockets; 2) most of the ATP binding site residues are visible and present in the crystal structures. These 89 protein kinase-inhibitor complexes include 25 different kinases, covering 14 different protein kinase subfamilies as classified by Hanks and Quinn. See Hanks and Hunter FASEB J 1995, 9, 576-596 and Hanks and Quinn Methods Enzymol., 1991, 200, 38-62. In all, the kinase structures contain 54 unique compounds representing a variety of chemical structures (see Table 1).

TABLE 1
List of 89 Crystal Structures of Protein Kinase-Ligand Complexes

| Protein | PDB accession | |
|---|---|---|
| Kinase | code | Ligand |
| Bovine PKA | 1ydt | H89 |
| | 1ydr | H7 |
| | 1yds | H8 |
| | 1stc | Staurosporine |
| Murine PKA | 1l3r | ADP |
| | 1fmo | Adenosine |
| | 1jbp | ADP |
| | 1atp | ATP |
| | 1bx6 | Balanol |
| Porcine PKA | 1cdk | AMPPNP |
| Human CDK2 | 1jvp | PKF049-365 |
| | 1fin | ATP |
| | 1jst | ATP |
| | 1e1v | NU2058 |
| | 1b38 | ATP |
| | 1jsv | U55 |
| | 1hck | ATP |
| | 1gy3 | ATP |
| | 1b39 | ATP |
| | 1gij | 2PU |
| | 1gii | 1PU |
| | 1gih | 1PU |
| | 1fq1 | ATP |
| | 1aq1 | Staurosporine |
| | 1ckp | Purvalanol |
| | 1e1x | NU6027 |
| | 1g5s | H717 |
| | 1fvt | 4-(5-BROMO-2-OXO-2H- |
| | | INDOL-3-YLAZO)- |
| | | BENZENESULFONAMIDE |
| | 1ke5 | LS1 |
| | 1ke9 | LS5 |
| | 1dm2 | Hymenialdisine |
| | 1fvv | 4-[(7-OXO-7H- |
| | | THIAZOLO[5,4-E]INDOL-8- |
| | | YLMETHYL)-AMINO]-N- |
| | | PYRIDIN-2-YL- |
| | | BENZENESULFONAMIDE |
| | 1di8 | 4-[3-HYDROXYANILINO]- |
| | | 6,7- |
| | | DIMETHOXYQUINAZOLINE |
| | 1e9h | INDIRUBIN-5-SULPHONATE |
| | 1ke8 | LS4 |
| | 1ke7 | LS3 |
| | 1ke6 | LS2 |
| S. pombe Ck-1 alpha | 1csn | ATP |
| | 2csn | CK17 |
| | 1eh4 | IC261 |
| Human c-src | 1byg | Staurosporine |
| | 1ksw | NBS |
| | 2src | AMPPNP |
| Human CK-2 alpha | 1jwh | AMPPNP |
| Human DAP | 1jkk | AMPPNP |
| | 1jkl | AMPPNP |
| | 1igl | AMPPNP |
| Human ERK2 | 1pme | SB202190 |
| Human FGFR | 2fgi | PD173074 |
| | 1agw | SU4984 |
| | 1fgi | SU5402 |
| Human HCK | 1ad5 | AMPPNP |
| | 1qcf | PP1 |
| | 2hck | Quercetin |
| Human IGFR | 1jqh | ACP |
| | 1k3a | ACP |
| Human INSR | 1i44 | AMPPNP |
| | 1ir3 | AMPPNP |
| | 1gag | Full-name |
| Human JNK3 | 1jnk | AMPPNP |
| Human LCK | 1qpd | Staurosporine |
| | 1qpe | PP2 |
| | 1qpj | Staurosporine |
| | 1qpc | AMPPNP |
| Human P38 | 1kv1 | BMU |
| | 1kv2 | BIRB-796 |
| | 1bmk | SB218655 |
| | 1bl7 | SB220025 |
| | 1di9 | 4-[3- |
| | | METHYLSULFANYLANILIN |
| | | O]-6,7- |
| | | DIMETHOXYQUINAZOLINE |
| | 1a9u | SB203580 |
| | 1bl6 | SB216995 |
| Murine ABL | 1iep | STI-571 |
| Murine ABL | 1fpu | STI-571 |
| Murine CHAK | 1iah | ADP |
| | 1ia9 | AMPPNP |
| Maize CK-2 alpha | 1lp4 | AMPPNP |
| | 1daw | AMPPNP |
| | 1j91 | TBS |
| | 1ds5 | AMP |
| | 1day | GNP |
| | 1f0q | Emodin |
| Murine NUK | 1jpa | AMPPNP |
| Human P38-gamma | 1cm8 | AMPPNP |
| Rat ERK2 | 1gol | ATP |
| | 4erk | Olomoucine |
| | 3erk | SB220025 |
| Rabbit PHK | 1phk | ATP |
| | 1q16 | ATP |
| | 2phk | ATP |

In each of Examples 1-3, the first step in the construction of SIFts is to identify a list of selected positions or binding site residues that are common in all complex structures being studied. The resulting panel of ligand binding site residues, which covered all of the interactions occurring between the target protein and the ligands, was then used as the common reference frame to construct the interactions fingerprints.

For a group of structures involving the same target protein (experiments such as those described in Examples 1 and 2), the ligand binding site is defined as the list of residues comprising the union of all residues involved in ligand binding over the entire library of structures. For a group of structures involving different target molecules (such as the experiment described in Example 3), additional structural and sequence pre-alignment steps were required as described immediately below.

In Example 3, the crystal structure of murine PKA complexed with ATP and a peptidic inhibitor PKI (PDB accession number: 1ATP; see Zheng et al. Acta Cryst. 1993, D49, 362-365) was used as the reference model for structural and sequence alignment. Initial amino acid sequence alignment of the catalytic cores of these kinases was taken from the Protein Kinase Resources (see Smith et al. TIBS, 1997, 22(11), 444-446). Structural alignment of the kinase structures was carried out manually and focused primarily on the vicinity of the ATP binding sites. Based on the structural alignment results, sequence alignments were carefully checked and adjusted if necessary, so that all structurally equivalent residues match each other in the sequence alignment. After the sequence and structural alignments, the residues of the non-murine PKA protein kinases were renumbered and tallied to the murine PKA residue numbering system, resulting in a uniform residue numbering system for all kinases analyzed. Identification of the list of ligand binding sites was carried out as previously described using the new PKA-equivalent residue numbers.

In each of Examples 1-3, after all the ligand binding site residues were identified and all the protein-ligand intermolecular interactions were calculated, the next step was to classify these interactions, as described previously in the “Detailed Description” Section. Seven different types of interactions occurring at each binding residue were extracted and classified from the AREAIMOL and HBPLUS results. The inquiries were: 1) whether or not it is in contact with the ligand; 2) whether or not any main-chain atom is involved in the contact; 3) whether or not any side-chain atom is involved in the binding; 4) whether or not a polar interaction is involved; 5) whether or not a non-polar interaction is involved; 6) whether or not the residue provides hydrogen bond acceptor(s); 7) whether or not it provides hydrogen-bond donor(s). By doing so, each residue was represented by a seven-bit-long bit string. The whole interaction fingerprint of the complex was finally constructed by sequentially concatenating the binding bit string of each binding site residue together, according to ascendant residue number order. Therefore, interaction fingerprints are of the same length and each bit in the fingerprint represents presence or absence of a particular interaction at a particular binding site.

As described above in Example 1, the SIFt-based method was applied to analyze the result of a typical docking study. This comprised of 100 docking poses of a small molecule inhibitor (SB203580) of p38, for which the crystal structure was known (PDB entry 1a9u). The poses adopted diverse binding modes, varied in their orientations and positions relative to the target protein and were complex to interpret visually (see FIG. 2A). A total of 34 protein residues in the vicinity of the ATP binding pocket were identified as the ligand binding site. These binding site residues were located in different sub-regions of the kinase structure. SIFts were generated for all complexes, each of which was composed of 238 (7×34) binary bits. The hierarchical clustering result of these fingerprints is shown in FIG. 2B with the fingerprint Tanimoto similarity matrix represented as a heat-map. The dendrogram revealed seven major clusters, labeled 1 to 7, respectively. FIG. 2B shows that the clustering by their SIFt patterns has separated the poses into different groups with distinct binding interactions. FIGS. 2C-2I depict the structures of each major cluster, each of which was put in the same reference frame. Interestingly, each of these seven clusters was comprised of poses having similar binding modes with the receptor. Cluster 1 contained molecules similar to the known X-ray crystal structure. Clusters 2-5 were similar in position but represented distinct binding modes that resulted in dissimilar interactions with the Gly-rich loop and the catalytic loop of p38. Finally, clusters 6 and 7 were outside the ATP binding site. Reassuringly, the degree of variation between clusters observed visually in their binding interactions appears to correlate to their distance in the dendrogram. For example, groups 1, 4, 6 and 7 each showed very little structural variation, as represented by tight clusters in the dendrogram, whereas group 3 and 5 showed relatively more diversity in their structures as well as in their fingerprints. Furthermore, clusters 1 and 7 had very little in common and were farthest from each other in the dendrogram. In summary, visual inspection confirms that SIFt is useful in separating docking poses into distinct clusters that reveal distinct binding interactions.

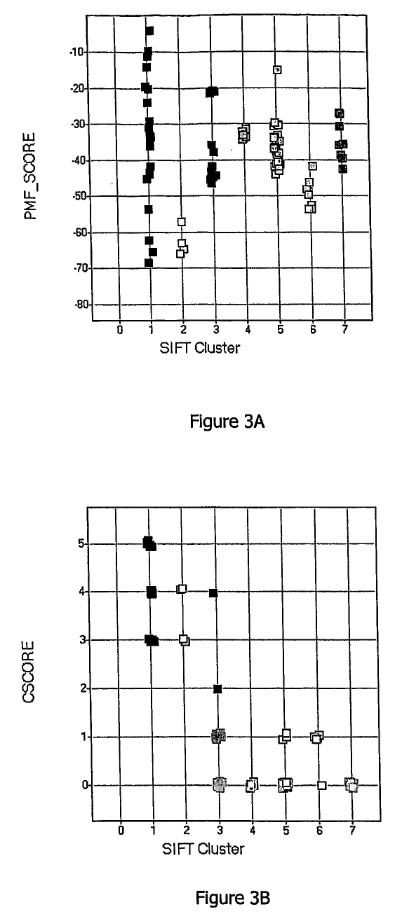

Traditionally, various scoring functions have been used to rank poses from docking studies. Scoring function scores provide an estimate of the binding strength of the compounds in order to identify the potential “good binders” from a large pool of poses, such that a selection of top scoring compounds derived from a rank ordered list of docked ligands will be enriched with active compounds. Scoring functions can be useful in discriminating the poses in the different SIFt clusters (i.e., different binding modes). In FIG. 3A, the first SIFt cluster, which is the closest to the true binding conformation, showed a wide range in PMF scores, spanning from the best score (−70) to the worst (−4). In fact, the majority of the poses in this cluster was no better in their PMF scores than those in other SIFt clusters. In addition, the PMF scores for SIFt cluster 2 were just as good as those for cluster 1, even though they adopt different, crystallographically unobserved, interactions with the receptor. Other different clusters also overlap with each other in their docking scores. Clearly, PMF score is a poor scoring function for discriminating compounds with true binding mode and irrelevant poses in the experiment. In an attempt to broaden the analysis of scoring functions, consensus scoring function that consists of five commonly used scoring functions was also examined (see FIG. 3B). Many of the poses in clusters 1-3 had high Cscores (3-5), while clusters 3-7 overlapped significantly in the score range 0-2. This example further demonstrates the fact that across a range of scoring functions, the energy-based approaches alone were insufficient in distinguishing different binding modes, and in isolating those poses corresponding to the observed binding mode.

The application of the SIFt-based method was extended to other ensembles of structures involving different proteins and a diverse set of small molecules. In Example 3, 89 known crystal structures of the protein kinase family that had been deposited in the Protein Databank were chosen. As mentioned above, they represent 14 different protein kinase subfamilies and 54 unique kinase small molecule ligands/inhibitors. The structure and sequence homology among protein kinases enabled us to analyze these structures using the SIFt-based approach.

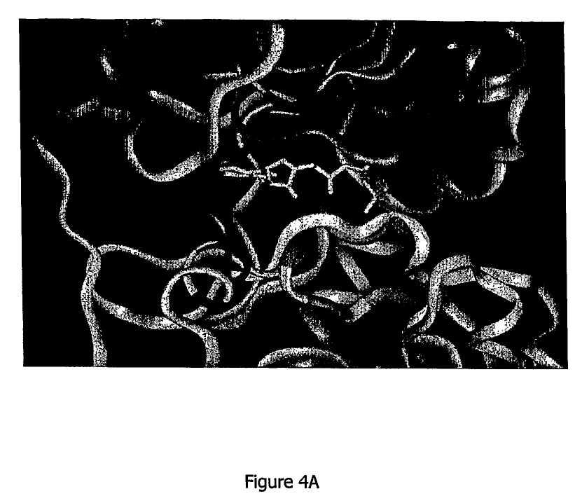

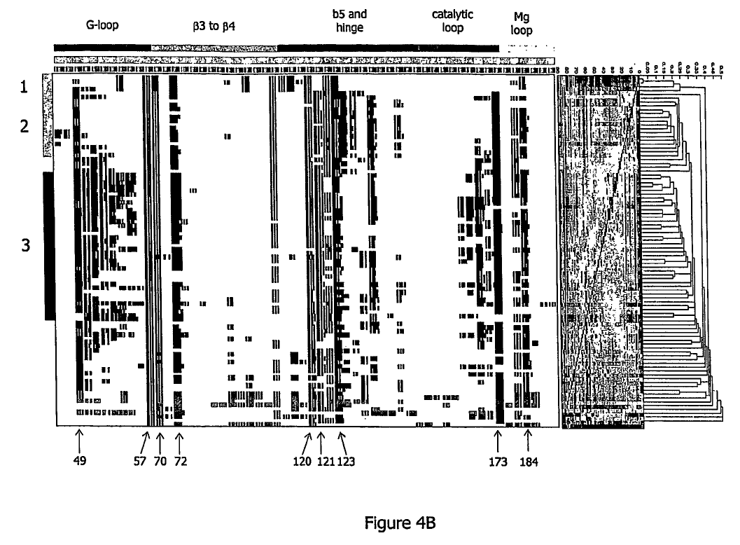

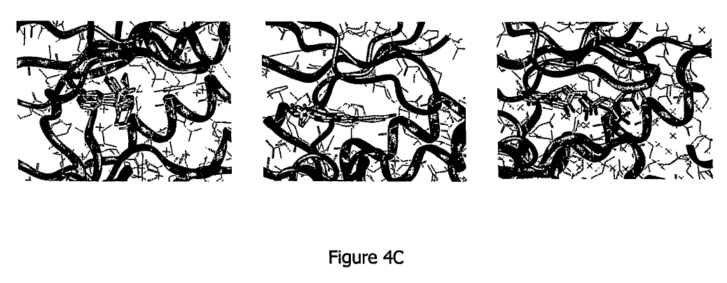

A total of 56 residues were identified as the ligandbinding site (see FIG. 4A). The heat-map and the results from hierarchical clustering are shown in FIG. 4B. These interaction fingerprints were diverse, reflecting a high degree of variability in their binding interactions. Nevertheless, three major clusters can be identified from the dendrogram (see FIG. 4B). Although the results indicate that within each cluster there existed considerable variation in their interaction patterns, these three groups represented three distinct binding modes, as confirmed by careful inspections of their structures (see FIG. 4C). The first cluster has 4 members, containing structures of human p38 in complex with four different pyridinyl imidazole inhibitors: SB203580, SB216995, SB220025 and SB218655. The second cluster had 16 members, mostly human CDK2 in complex with different compounds with diverse chemical properties. The third cluster, which does not have a clear-cut boundary, is comprised of approximately 36 structures, and almost all of them are structures of different kinases in complex with ATP or ATP-analogs inhibitors (GTP, AMPPNP, AMPPCP, AMP, ADP, etc.). Besides these three major clusters, about one-third of the 89 structures are either singletons or form tiny clusters. Interestingly, the three major clusters represent different grouping examples of protein-ligand complexes—the first one is made up of the same protein and chemically similar compounds; the second group contains the same protein but with a variety of ligands; the third cluster contains different proteins in complex with chemically similar ligands.

Comparison of these fingerprints also revealed interactions that are conserved or highly variable among the structures. For instance, contact interactions with residue 57 (in PKA numbering, within the Gly-rich loop) and residue 70 (also in PKA numbering), are strictly conserved among all of the 89 protein kinase-ligand structures. Other highly conserved interactions include contacts with residue 49, 72, 120, 121, 123, 173, 184, etc. (see FIG. 4B). In contrast, many other interactions are not conserved or only conserved within a particular group. Detailed and systematic comparison of these structural profiles of the ATP binding sites of protein kinases will be presented elsewhere (Deng et al. manuscript in preparation).

The SIFt-based method provides a new and powerful tool for lead discovery and lead optimization, enabling the search for molecules in a chemical database on the basis of expected interaction patterns to a target molecule. This application was specifically tested in Example 2, where a virtual screen for a set of 16 known p38 inhibitors spiked into a diverse library of 1,000 commercially available compounds was performed. These p38 inhibitors were all ATP-competitive inhibitors, and despite representing varied chemical templates had similarities to the pyridinylimidazole series (i.e., SB203580-like) for which the crystal structure of the complex was known (1a9u).

These inhibitors and the random collection of chemical compounds were docked using FlexX onto the crystal structure of p38 (1a9u), and how well these known inhibitors could be enriched using commonly used scoring functions was assessed. These were then compared with the results from a SIFt-based enrichment involving filtering of the compounds based on their similarities in interaction patterns (measured by Tanimoto coefficient) to SB203580, a known pyridinylimidazole inhibitor of p38 for which the X-ray crystal structure was known. The rationale for SIFt-based enrichment is that these 16 known inhibitors, being analogs of the pyridinylimidazole series, are expected to bind to p38 with similar overall binding modes.

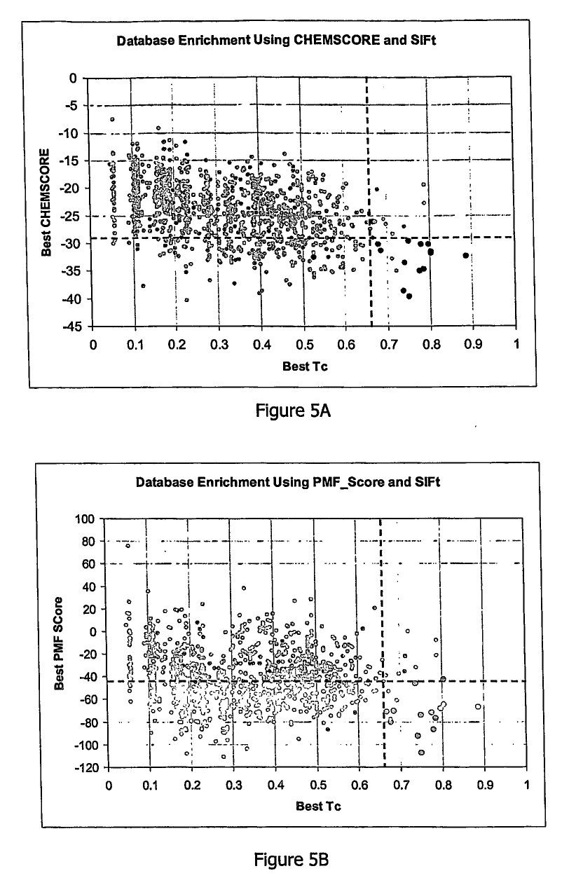

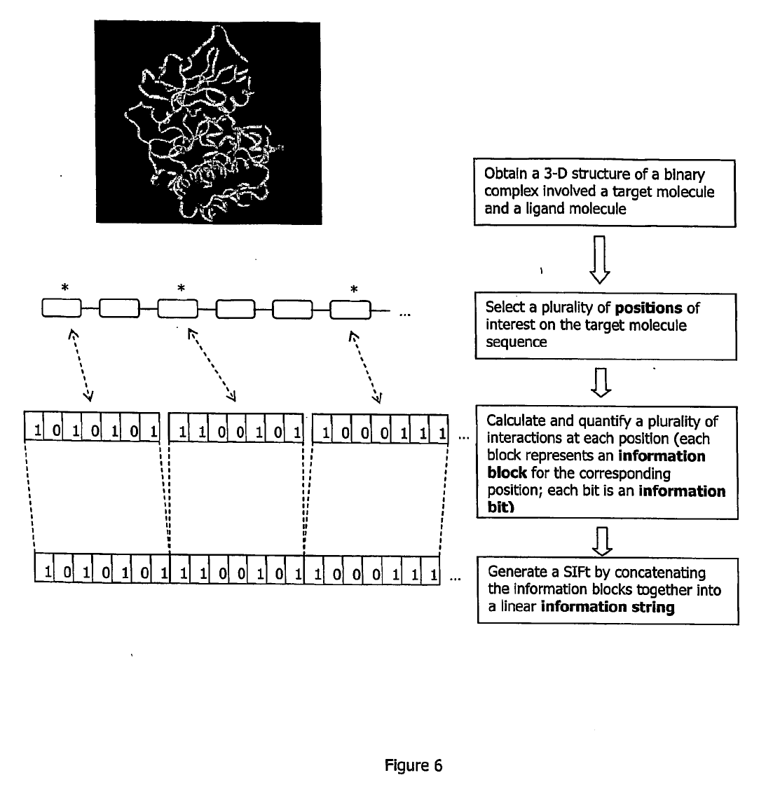

FIG. 5A, 5B and Table 1 show the comparison of the database enrichment performances of the scoring functions with SIFt. ChemScore gave a modest enrichment factor of 5.4, and 166 compounds were harvested in order to identify 14 of the 16 known p38 inhibitors. PMF was slightly worse than ChemScore, with an enrichment factor of 2.0. In addition, an analysis of the binding modes of the poses of the enriched p38 inhibitors identified using these scoring functions showed that some of them were highly variable to the known crystal structure of SB203580, despite similarities in functionalities, suggesting that their binding modes obtained by ChemScore or PMF score were incorrect. This implies that the scoring functions were probably performing worse than the enrichment factors were indicating. In comparison, SIFt scored quite well, having to harvest only 24 compounds to be able to identify 14 of the 16 inhibitors, giving an enforcement factor of 37.0. Reassuringly, the highest scoring compound recovered by SIFt was SB203580 upon which the interaction fingerprint used to probe the database was based. Visual inspection of the binding modes of the p38 inhibitors identified using SIFt showed that all of their binding modes were similar to that of SB203580. A combination of SIFt and ChemScore led to a modest increase in enrichment (EF=42.3).

TABLE 2
Comparison of the database enrichment performances
of SIFt with ChemScore and PMF Score

| Filtering Method | Enrichment Factor (EF)* |
|---|---|
| PMF Score | 2.0 |
| ChemScore | 5.4 |
| SIFt | 37.0 |
| SIFt + ChemScore | 42.3 |

*EF is defined as: EF = {Hits_{sampled}/N_{sampled}}/{Hitsₜₒₜₐₗ/Nₜₒₜₐₗ}, where Hits_{sampled} is the number of known inhibitors recovered the sampled fraction of N_{sampled} poses; Hitsₜₒₜₐₗ is the number of known inhibitors present in the whole library of Nₜₒₜₐₗ compounds¹⁴. Here each EF was calculated based on the ability of recovering 14 out of 16 known p38 inhibitors spiked into a random library of 1,000 compounds.

### Example 4 and 5

These two examples illustrate two other embodiments of SIFt implementation that include the chemical information about the ligands into their SIFt patterns. In Example 4, the information about core and variable groups (R-groups) of a compound is embedded into the SIFts (e.g., R-SIFts); in Example 5, the pharmacophoric features of the compound are used.

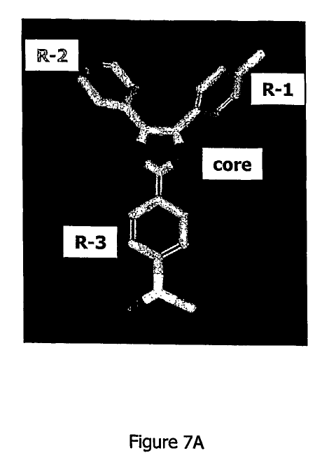

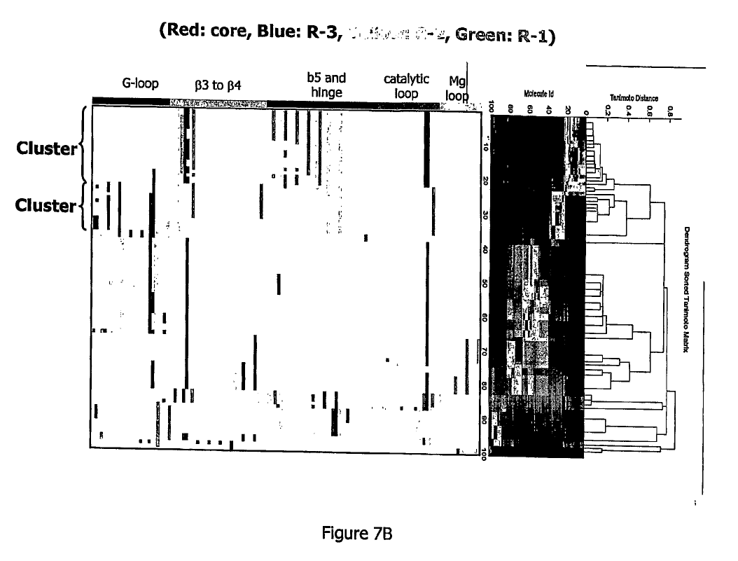

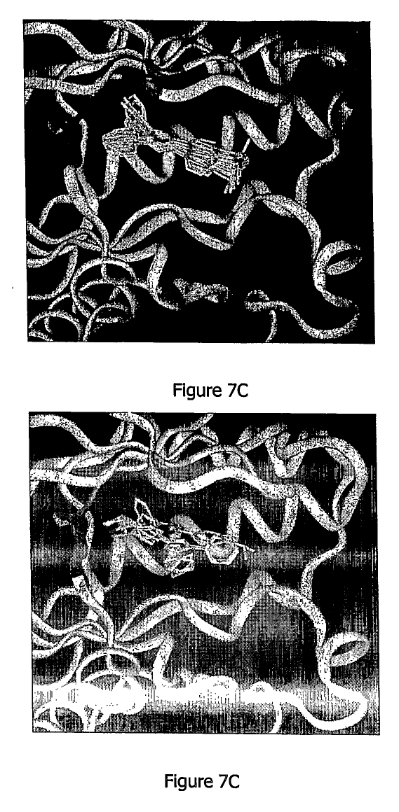

In Example 4, the same set of 100 docking poses of SB203580 docked onto p38 used in Example 1 and 2 was also used. The SB203580 molecule was decomposed into core, R1, R2 and R3 groups as shown in FIG. 7A. Each non-hydrogen atoms were assigned to one of these four different groups. Four binary bits were used for each binding site residue, representing the core, R-1, R-2, R-3, respectively. If this residue was in contact with (i.e., distance <=4.0 Angstrom) a non-hydrogen atom belonging to a particular group, then the corresponding bit is turned ON (1); otherwise the bit remains OFF (0). The final SIFt pattern was constructed by concatenating all the bit strings of all the binding site residues together, according to the same ascendant residue number order, as used in Example 1.

Grouping of the SIFt patterns was carried out using the same hierarchical clustering method as described in Example 1.

FIG. 7A is the decomposition of molecule SB203580 into core (1) and three different R-groups, R-1 (2), R-2 (3) and R-3 (4).

FIG. 7B is a hierarchical clustering of the SIFts of 100 SB203580 docking poses. The SIFts were constructed to represent different R-groups and the core of the molecule. Each selected position of the target molecule is made up of four binary bits, representing core, R1, R2, R3, and R4, respectively. Each SIFt was shown as one line in the heat map in the left of the figure, and only ON-bits are shown. The shades of gray, or colors, of the heat map blocks indicated different R-groups: red—core, blue—R-1, yellow —R-2, green—R-3. On the right side of the figure showed the hierarchical clustering results on the fingerprints, including the dendrogram and the reorganized distance matrix. SIFts in the heat map were reorganized according to the order given by the hierarchical clustering. The shaded, or colored, bar on top of the SIFt heat map represents five kinase sub-regions in the fingerprints. These sub-regions, each shaded or colored differently, include the Gly-rich loop (G-loop), the region spanning from β3 to β4 (β3 to β4), β5 and the hinge region, catalytic loop and magnesium loop.

FIG. 7C and 7D show the structures of the poses in cluster 1 (7C) and cluster 2 (7D), respectively, as identified by the hierarchical clustering of their R-SIFts (FIG. 7B), in the context of the p38 crystal structure (1a9u). The poses are shown in gray or cyan, and the co-crystal structure of SB203580 is shaded or colored according to atom types. The five kinase sub-regions that are in contact with the poses within the group are shaded or colored using the same shading or coloring scheme as described in FIG. 2B and FIG. 7B. Compared to Example 1, the 7 R-SIFt groups are more tightly clustered, indicating R-SIFt is more sensitive to the different binding mode than the original SIFt comprised of 7 interaction bits that were used in Example 1. In addition, since different bits in the R-SIFt correspond to different segments of the molecule, it is very straightforward to tell from the R-SIFt which part of the molecule interacts with which part of the target molecule. Therefore, R-SIFt can be used in virtual screening as a convenient tool to separate poses of different binding modes.

In Example 5, the same set of SB203580 docking poses were used. This time, however, each atom of the molecule was assigned to seven different chemical features, including hydrogen bond acceptor, hydrogen bond donor, hydrophobic, polar, negatively charged, positively charged, or aromatic ring atom. Some atoms fell into more than one category of these chemical features. When constructing the new SIFt patterns, seven binary bits were used to represent a binding site residue, each indicating one of the above seven chemical features. If this residue was within 4.0 Angstroms from any atom that belongs to a particular chemical feature category, then this bit was turned ON (1); otherwise it remained OFF (0). The final SIFt was constructed by concatenating all the binary strings for all binding site residue together, in the same order as used in Examples 1 and 4.

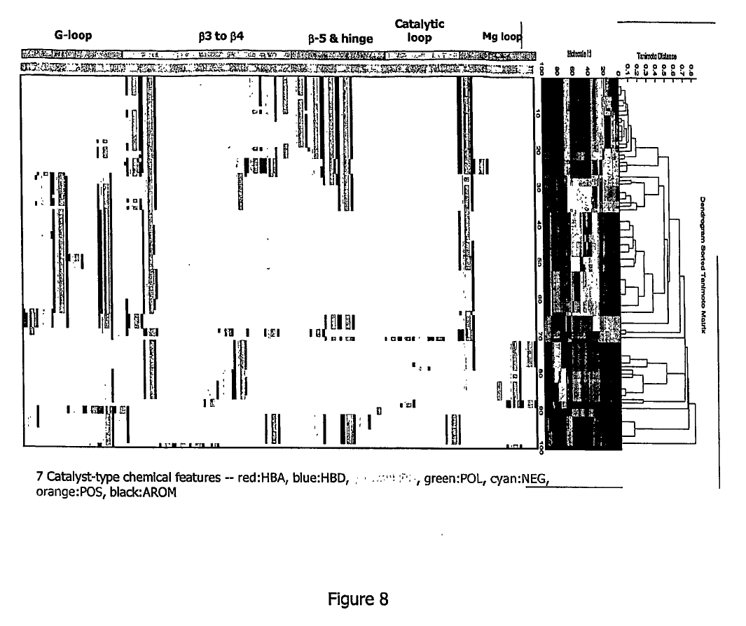

FIG. 8 is the hierarchical clustering of the SIFts of the same 100 docking poses of SB203580. Here the SIFt patterns contained 7 bits per selected position, each representing one of the seven chemical features of the molecule: red—hydrogen bond acceptor, blue—hydrogen bond donor, yellow—hydrophobic, green—polar, cyan—negatively charged, orange—positively charged, black—aromatic ring. These colors are represented in shades of gray in FIG. *. The hierarchical clustering was based on the new SIFt patterns incorporating the chemical features of the molecules.

In both Examples 4 and 5, the two different constructions of SIFt pattern provided richer information about the chemical environment around the binding site. Hierarchical clustering results of these two set of new SIFts both gave similar performance, in terms of separating different binding modes of the poses, and the results were comparable with that given by the previous construction of SIFt described in Example 1. This indicates that both the SIFt patterns incorporating the information about the R-group and chemical features were very useful ways of representing the structural information, complimentary to the previous construction of SIFt.

### Example 6

This example demonstrates one of many potential applications of the interaction profile. A structural interaction profile represents the degree of similarity for an interaction occurring at a particular binding site among a group of structures. In this example, the value at each position is the average of all the interaction bit values occurring at this particular position within a group of SIFts.

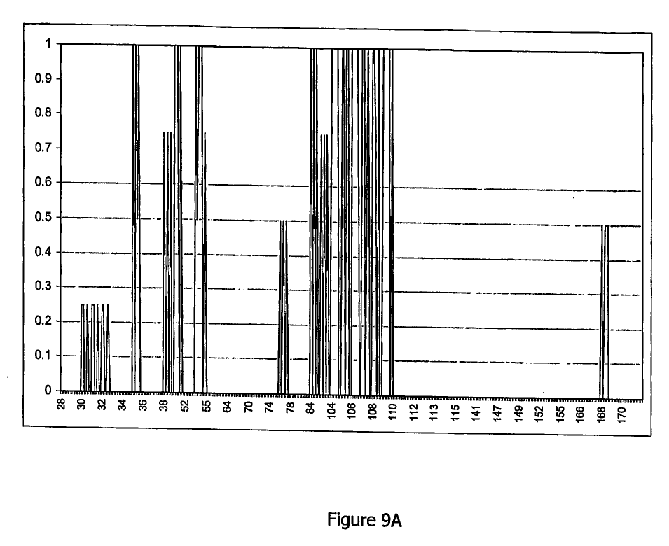

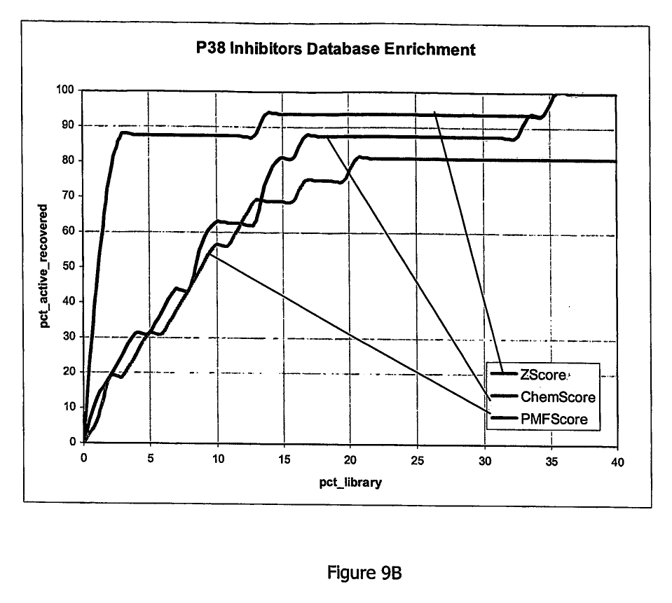

FIG. 9A shows the interaction profile generated from the SIFt patterns of four p38 crystal structures—1a9u, 1b16, 1b17, and 1bmk, each of which contains a different potent p38 inhibitor. The X-axis represents the p38 residue numbers of the interaction bits; the Y-axis represents the conservation scores of the interaction bits. The more conserved an interaction, the higher the value at this position.

The above interaction profile was used to enrich p38 inhibitors from a large library. The idea behind the approach is that if a compound adopts an interaction pattern similar to that of previously known inhibitors (i.e., an interaction profile), then it is more likely to be a true inhibitor. The statistical Z score was used to measure how significant the similarity between a SIFt and a target profile is above a certain background. Z score is defined as

$$Z = \frac{x - < {x}_{b} >}{{σ}_{b}}$$

where x is the Tanimoto coefficient of the SIFt against the target profile, <x_{b}> and a are the mean and standard deviation of the Tanimoto coefficients of all the SIFts in the background set, respectively, against the same target profile. The background set was used to construct a reference distribution upon which the comparisons were based.

A library comprised of sixteen known p38 inhibitors and 1000 random compounds were docked onto p38 target molecule. For each compound, 10 poses were retained for subsequent analysis. Poses were ranked according to their SIFt Z scores against the p38 interaction profile, generated from four co-crystal structures. The background set used in Z score calculation included all of the docking poses. For each compound, the pose with the highest Tanimoto coefficient against the p38 profile was selected, and then all 1016 best poses were ranked according to their Z score. The database enrichment curves are shown in FIG. 9B. The X-axis is the percentage of the whole library collected, and the Y-axis is the percentage of active compounds harvested. For comparison, the enrichment performances by two conventional scoring functions (ChemScore and PMF Score) are also shown.

From FIG. 9B it is clear that the enrichment obtained by applying SIFt-based Z score to select the best pose for each compound provided markedly superior results over those obtained using standard scoring using the ChemScore and PMF Score.

A number of embodiments have been described. Nevertheless, it will be understood that various modifications may be made. Accordingly, other embodiments are within the scope of the following claims.

What is Claimed is:

1. A method for generating a structural interaction fingerprint (SIFt) in the form of an information string which comprises a plurality of information blocks wherein each information block comprises a plurality of information units, the method comprising:
selecting a plurality of selected positions on a target molecule, wherein each selected position corresponds to an information block in the information string, the target molecule forming a complex with a ligand;
selecting a plurality of interaction types and calculating a value that is indicative of a characteristic of each interaction type at each selected position of the target molecule;
assigning the value to a corresponding information unit, the information unit indicating a characteristic of the interaction type at the corresponding selected position;
joining the information units of each selected position together to form corresponding information blocks; and
joining the information blocks together to generate a SIFt.

2. The method of claim 1, wherein the target molecule is a protein or a peptide.

3. The method of claim 1, wherein the target molecule is a nucleic acid.

4. The method of claim 1, wherein the ligand is a small molecule, a peptide, a protein or a nucleic acid.

5. The method of claim 1, wherein the value that is assigned to an information unit is a binary value, which indicates the presence or absence of a particular interaction type at the corresponding selected position.

6. The method of claim 1, wherein the value that is assigned to an information unit is a numeric value selected from a scale of numbers, wherein the numeric value indicates the magnitude of a particular interaction type at the corresponding selected position.

7. The method of claim 1, wherein the selected positions are obtained from a three-dimensional structure of a binary complex formed between the target molecule and the ligand.

8. The method of claim 7, wherein the three-dimensional structure is derived from an experimental method or a prediction method.

9. The method of claim 2, wherein each selected position comprises one or more secondary structure elements, amino acid residues, main chain atom groups, side chain atom groups, or individual atoms of the target molecule.

10. The method of claim 3, wherein each selected position comprises one or more bases, functional groups, or individual atoms of the target molecule.

11. The method of claim 1, wherein the interaction types represent different types of intermolecular interactions between the target molecule and the ligand.

12. The method of claim 1, wherein the interaction types is selected from the group consisting of contact interaction, polar interaction, non-polar interaction, or hydrogen bonding interaction.

13. The method of claim 11, wherein the intermolecular interactions are characterized by interaction energy-based approach.

14. The method of claim 12, wherein the contact interaction comprises an inter-atomic contact distance between the target molecule and the ligand of less than 10 Å.

15. The method of claim 12, wherein the contact interaction comprises an inter-atomic contact distance between the target molecule and the ligand of less than 6 Å.

16. The method of claim 12, wherein the contact interaction comprises a change in the accessible surface area of the target molecule upon forming a complex with the ligand.

17. The method of claim 12, wherein the hydrogen bonding interaction comprises a hydrogen bond donor in the target molecule and a hydrogen bond acceptor in the ligand at the selected position.

18. The method of claim 12, wherein the hydrogen bonding interaction comprises a hydrogen bond acceptor in the target molecule and a hydrogen bond donor in the ligand at the selected position.

19. The method of claim 1, wherein at least one interaction type includes a chemical or physical property of a part of ligand interacting with each selected position.

20. The method of claim 1, wherein each interaction type includes a chemical and physical property of a part of ligand interacting with each selected position.

21. The method of claim 19, wherein interaction types include information bits representing a core of a combinatorial library.

22. The method of claim 19, wherein interaction types include information bits representing a varying group of a combinatorial library.

23. The method of claim 19, wherein the interaction type includes a chemical property selected from the group consisting of hydrogen bond acceptor, hydrogen bond donor, hydrophobic, hydrophobic aliphatic, hydrophobic aromatic, negative charge, negative ionizible, positive charge, positive ionizible, or aromatic ring.

24. The method of claim 19, wherein the interaction type is an experimentally determined or computed property of the part of the ligand interacting with the selected position.

25. The method of claim 19, wherein the interaction type includes a chemical fingerprint for a part of the ligand interacting with the selected position of the target molecule.

26. The method of claim 1, wherein at least one interaction type includes a variable measuring the sequence conservation, structural conservation and flexibility of the selected position of the target molecule.

27. The method of claim 1, wherein the first ligand is the natural ligand of the target molecule or a ligand of known affinity to the target molecule.

28. The method of claim 1, further comprising calculating a score for each interaction among the target molecule-ligand complexes.

29. The method of claim 1, further comprising compiling the SIFts to generate an interaction profile from the calculated scores, wherein the interaction profile is indicative of the degrees of similarity of each information bit within the SIFts.

30. The method of claim 1, further comprising comparing a SIFt generated from a test ligand with an interaction profile generated from a group of target molecule-ligand complexes, thereby predicting whether the test ligand interacts with the target molecule in a similar pattern with the group.

31. The method of claim 1, further comprising comparing two interaction profiles, thereby predicting whether two groups of structures share conserved binding interactions, and/or have similar binding pattern.

32. A method of predicting the interaction pattern between a target molecule and a test ligand, the method comprising:
identifying a plurality of selected positions between the target molecule and a first ligand, wherein the first ligand is known to bind to the target molecule;
generating a first structural interaction fingerprint (SIFt);
generating a second SIFt between the target molecule and a second ligand (test ligand) by repeating the identifying step and the generating a first SIFt step using the second ligand; and
comparing the first SIFt with the second SIFt to determine a level of overlapping, wherein a level of substantial overlapping predicts that each of the first ligand and the second ligand interacts with the target molecule in a similar pattern.

33. A method of generating a structural interaction fingerprint (SIFt) database, the method comprising:
identifying a plurality of selected positions on a target molecule which is forming a complex with a first ligand;
generating a first SIFt of the database;
repeating the identifying step and the generating a first SIFt step using the same target molecule and a different ligand to generate a further SIFt of the database; and
repeating the repeating step until the database contains a desired number of SIFts.

34. The method of claim 33, further comprising analyzing the SIFts of the database to generate one or more interaction patterns between the target molecule and the ligands.

35. The method of claim 34, further comprising comparing one interaction pattern of the database with a SIFt generated by using the same target molecule and a test ligand, thereby predicting whether said test ligand belongs to the family of ligands used to generate the database.

36. The method of claim 33, further comprising storing the database in a computer readable medium.

37. A method of analyzing the interaction pattern of two or more related target molecules, said method comprising:
conducting sequence and structural alignments among each of the related target molecules to derive a uniform residue or base numbering system;
identifying a plurality of selected positions on the target molecule of each target molecule-ligand complex using one of the uniform residue or base numbering system;
generating a SIFt for each target molecule-ligand complex; and
comparing different SIFt patterns.

38. The method of claim 37, wherein at least one of the selected interactions among the complexes is conserved.

39. The method of claim 37, wherein at least one of the selected interactions among the complexes is unconserved.

40. The method of claim 37, wherein the related target molecules have at least 20% sequence similarity or a structural similarity with a root-mean squared deviation over the aligned positions no greater than 6 Å.

41. The method of claim 37, wherein the related target molecules have at least 20% sequence identity or a structural similarity with a root-mean squared deviation over the aligned positions no greater than 4 [symbol] root-mean squared deviation over the aligned positions.

42. A computer-readable data storage medium comprising a data storage material encoded with a computer-readable database, the database comprising a plurality of structural interaction fingerprints (SIFts) generated from a target molecule and a plurality of ligands, wherein each SIFt is in the form of an information string comprising a plurality of information blocks and each information block comprising a plurality of information units, wherein said target molecule interacts with each ligand at a plurality of selected positions on the target molecule using a number of interaction types, and wherein the magnitude of each interaction type at each selected position is calculated and represented by a value which is assigned to an information unit.

43. The computer-readable data storage medium of claim 42, wherein the target molecule is a protein or a peptide.

44. The computer-readable data storage medium of claim 42, wherein the target molecule is a nucleic acid.

45. The computer-readable data storage medium of claim 42, wherein the ligand is a small molecule, a peptide, or a nucleic acid.

46. The computer-readable data storage medium of claim 42, wherein the value that is assigned to an information unit is a binary value, which indicates the presence or absence of a particular interaction type at the corresponding selected position.

47. The computer-readable data storage medium of claim 42, wherein the value that is assigned to an information unit is selected from a range of scaled numeric values, which indicates the magnitude of a particular interaction type at the corresponding selected position.

48. The computer-readable data storage medium of claim 42, wherein each selected position comprises one or more amino acid residues, main chain atom groups, side chain atom groups, or individual atoms of the target molecule.

49. The computer-readable data storage medium of claim 42, wherein each selected position comprises one or more bases, functional groups, or individual atoms of the target molecule.

50. The computer-readable data storage medium of claim 42, wherein each interaction type is selected from the group consisting of contact interaction, polar interaction, non-polar interaction, and hydrogen bond interaction.

51. The computer-readable data storage medium of claim 50, wherein the contact interaction comprises an inter-atomic contact distance between the target molecule and the ligand of less than 10 Å.

52. The computer-readable data storage medium of claim 50, wherein the contact interaction comprises an inter-atomic contact distance between the target molecule and the ligand of less than 6 Å.

53. The computer-readable data storage medium of claim 50, wherein the contact interaction comprises a change in the accessible surface area of the target molecule upon forming a complex with the ligand.

54. The computer-readable data storage medium of claim 50, wherein the hydrogen bond interaction comprises a hydrogen bond donor in the target molecule and a hydrogen bond acceptor in the ligand at the corresponding selected position.

55. The computer-readable data storage medium of claim 50, wherein the hydrogen bond interaction comprises a hydrogen bond acceptor in the target molecule and a hydrogen bond donor in the ligand at the corresponding selected position.

56. A computer program for generating a structural interaction fingerprint (SIFt) in the form of an information string which comprises a plurality of information blocks, wherein each information block comprises a plurality of information units, the computer program comprising instructions for causing a computer system to:
select a plurality of selected positions on a target molecule wherein each selected position corresponds to an information block in the information string, the target molecule forming a complex with a ligand;
select a plurality of interaction types and calculate a value that is indicative of the characteristic of each interaction type at each selected position of the target molecule;
assign the value to a corresponding information unit, the information unit indicating a characteristic of the interaction type at the corresponding selected position;
join the information units of each selected position together to form corresponding information blocks; and
join the information blocks to generate a SIFt.

57. The computer program of claim 56, wherein the target molecule is a protein or a peptide.

58. The computer program of claim 56, wherein the target molecule is a nucleic acid.

59. The computer program of claim 56, wherein the ligand is a small molecule, a peptide, or a nucleic acid.

60. The computer program of claim 56, wherein the value that is assigned to an information unit is a binary value indicating the presence or absence of a particular interaction type at the corresponding selected position.

61. The computer program of claim 56, wherein the value that is assigned to an information unit is a numeric value selected from a scale of numbers, wherein the numeric value indicates the magnitude of a particular interaction type at the corresponding selected position.

62. The computer program of claim 56, wherein the selected positions are obtained from a three-dimensional structure of a binary complex formed between the target molecule and the ligand.

63. The computer program of claim 62, wherein the three-dimensional structure is derived from an experimental method or a prediction method.

64. The computer program of claim 57, wherein each selected position comprises one or more secondary structure elements, amino acid residues, main chain atom groups, side chain atom groups, or individual atoms of the target molecule.

65. The computer program of claim 58, wherein each selected position comprises one or more bases, functional groups, or individual atoms of the target molecule.

66. The computer program of claim 56, wherein the interaction types represent different types of intermolecular interactions between the target molecule and the ligand.

67. The computer program of claim 56, wherein the interaction types is selected from the group consisting of contact interaction, polar interaction, non-polar interaction, or hydrogen bonding interaction.

68. The computer program of claim 66, wherein the intermolecular interactions are characterized by interaction energy-based approach.

69. The computer program of claim 67, wherein the contact interaction comprises an inter-atomic contact distance between the target molecule and the ligand of less than 10 Å.

70. The computer program of claim 67, wherein the contact interaction comprises an inter-atomic contact distance between the target molecule and the ligand of less than 6 Å.

71. The computer program of claim 67, wherein the contact interaction comprises a change in the accessible surface area of the target molecule upon forming a complex with the ligand.

72. The computer program of claim 67, wherein the hydrogen bonding interaction comprises a hydrogen bond donor in the target molecule and a hydrogen bond acceptor in the ligand at the selected position.

73. The computer program of claim 56, further comprising instructions to store the SIFt in a database.

74. The computer program of claim 67, further comprising instructions to store the SIFts in a database.

75. The computer program of claim 56, further comprising instructions to
generate a further SIFt between the target molecule and a second ligand without changing the selected positions and the interaction types; and
comparing the original SIFt with the further SIFt to determine a level of overlapping, wherein a pattern of substantial overlapping predicts that each of the original ligand and the second ligand interacts with the target molecule in a similar pattern.

76. The computer program of claim 75, wherein the original ligand is the natural ligand of the target molecule.
